SEQUENCE LISTING (20 sequences)

SEQ ID NO: 1 - protein, 444 aa, synthetic construct
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, synthetic construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 19 aa, synthetic construct
LSCAASGYSFTGYYIHWVR

SEQ ID NO: 4 - protein, 7 aa, synthetic construct
GLEWVAR

SEQ ID NO: 5 - protein, 13 aa, synthetic construct
VIPNAGGTSYNQK

SEQ ID NO: 6 - protein, 8 aa, synthetic construct
FTLSVDNS

SEQ ID NO: 7 - protein, 11 aa, synthetic construct
NTAYLQMNSLR

SEQ ID NO: 8 - protein, 20 aa, synthetic construct
EGIYWWGQGTLVTVSSASTK

SEQ ID NO: 9 - protein, 23 aa, synthetic construct
SSQSLVHSNGNTFLHWYQQKPGK

SEQ ID NO: 10 - protein, 9 aa, synthetic construct
LLIYTVSNR

SEQ ID NO: 11 - protein, 7 aa, synthetic construct
FSGVPSR

SEQ ID NO: 12 - protein, 21 aa, synthetic construct
PGKAPKLLIYTVSNRFSGVPS

SEQ ID NO: 13 - protein, 113 aa, synthetic construct
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVS

SEQ ID NO: 14 - protein, 113 aa, synthetic construct
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKR

SEQ ID NO: 15 - protein, 10 aa, synthetic construct
GYSFTGYYIH

SEQ ID NO: 16 - protein, 16 aa, synthetic construct
RVIPNAGGTSYNQKFK

SEQ ID NO: 17 - protein, 8 aa, synthetic construct
YCAREGIY

SEQ ID NO: 18 - protein, 12 aa, synthetic construct
RSSQSLVHSNGN

SEQ ID NO: 19 - protein, 7 aa, synthetic construct
KLLIYTV

SEQ ID NO: 20 - protein, 9 aa, synthetic construct
ATYFCSQTT

(19) United States
(12) Patent Application Publication
(10) Pub. No.: US 2024/0142463 A1
(43) Pub. Date: May 2, 2024

(54) METHODS OF QUANTIFYING ANTI-CD40 ANTIBODIES

(71) Applicant: Seagen Inc., Bothell, WA (US)

(72) Inventors: Shawna Hengel, Shoreline, WA (US); Steve Alley, Seattle, WA (US); Barry Jones, Durham, NC (US)

(21) Appl. No.: 18/516,634

(22) Filed: Nov. 21, 2023

Related U.S. Application Data
(63) Continuation of application No. PCT/US22/30850, filed on May 25, 2022.
(60) Provisional application No. 63/192,846, filed on May 25, 2021.

(51) Int. Cl. G01N 33/68

(52) U.S. Cl. CPC G01N 33/6848; G01N 33/686; G01N 2333/95; G01N 2440/38; G01N 2458/15

(57) ABSTRACT

The present disclosure relates to methods of determining the amount of an anti-CD40 antibody in a sample. The present disclosure also relates to anti-CD40 antibody signature analytic peptides.

### CROSS-REFERENCE TO RELATED APPLICATIONS

Pursuant to 35 U.S.C. § 119(e), this application is a continuation of International Application PCT/US2022/030850, with an international filing date of May 25, 2022, which claims the benefit of U.S. Provisional Patent Applications Nos. 63/192,846, filed on May 25, 2021, the contents of which are hereby incorporated by reference herein in its entirety.

### SEQUENCE LISTING

This application contains a Sequence Listing that has been submitted electronically as an XML file named 49223-0055001 SL ST26.xml. The XML file, created on Nov. 20, 2023, is 19,234 bytes in size. The material in the XML file is hereby incorporated by reference in its entirety.

### TECHNICAL FIELD

The present disclosure relates to methods of quantifying anti-CD40 antibodies.

### BACKGROUND

An intense research effort is directed towards improving pharmacokinetic profiles, toxicity and chemical stability of therapeutic antibodies. Pharmacokinetic profiles may be assayed by measuring the concentration of the therapeutic antibody over time, or after exposure to the biological environment using various analytic methods. However, current assays for quantifying therapeutic antibodies have background interference and limitations in sensitivity and selectivity, which can vary from sample to sample or patient to patient. Therefore, there is a need for alternative means of quantifying therapeutic antibodies.

### SUMMARY OF THE INVENTION

The present disclosure relates to methods for quantifying an anti-CD40 antibody in a sample with improved sensitivity and selectivity. In one aspect, after extracting the anti-CD40 antibody from a sample, an analytic peptide is released from the anti-CD40 antibody, such that measurement of the analytic peptide is equivalent to a measurement of the anti-CD40 antibody and can be used to determine the amount of anti-CD40 antibody in the sample.

In one aspect, the disclosure provides a method for determining the amount of an anti-CD40 antibody or antigen-binding fragment thereof in a sample. In some embodiments, the anti-CD40 antibody or antigen-binding fragment thereof comprises: a heavy chain variable region (VH) comprising an amino acid sequence of SEQ ID NO: 13; and a light chain variable region (VL) comprising an amino acid sequence of SEQ ID NO: 14. In some embodiments, the method comprises extracting the anti-CD40 antibody or antigen-binding fragment thereof from the sample; digesting the extracted anti-CD40 antibody or antigen-binding fragment thereof with a protease to release one or more analytic peptides from the anti-CD40 antibody or antigen-binding fragment thereof; and detecting the one or more analytic peptides by mass spectrometry, thereby determining the amount of the anti-CD40 antibody or antigen-binding fragment thereof in the sample.

In some embodiments, the anti-CD40 antibody or antigen-binding fragment thereof is extracted by anti-idiotypic affinity capture. In some embodiments, the extracted anti-CD40 antibody or antigen-binding fragment thereof is digested by either one or both of trypsin and Lys-C. In some embodiments, the extracted anti-CD40 antibody or antigen-binding fragment thereof is digested in a digestion solution comprising about 10 μg/mL to about 100 μg/mL of trypsin and/or Lys-C. In some embodiments, the concentration of trypsin and/or Lys-C in the digest solution is about 50 μg/mL In some embodiments, the digestion solution further comprises about 0.25 M to about 1.0 M Tris, with a pH of about 8.0 to about 8.5. In some embodiments, the extracted anti-CD40 antibody or antigen-binding fragment thereof is not subject to either reduction or alkylation. In some embodiments, the extracted anti-CD40 antibody or antigen-binding fragment thereof is eluted from the anti-idiotypic affinity capture with an elution solution comprising about 10 mM to about 50 mM hydrochloric acid (HCl). In some embodiments, the extracted anti-CD40 antibody or antigen-binding fragment thereof is eluted from the anti-idiotypic affinity capture with an elution solution comprising about 30 mM hydrochloric acid (HCl).

In some embodiments, the anti-CD40 antibody comprises a human constant region. In some embodiments, the anti-CD40 antibody comprises a heavy chain with a sequence set forth in SEQ ID NO: 1 and a light chain with a sequence set forth in SEQ ID NO: 2. In some embodiments, the anti-CD40 antibody is non-fucosylated. In some embodiments, the anti-CD40 antibody is SEA- CD40. In some embodiments, less than 5% of the anti-CD40 antibody in the sample has an N-glycoside-linked sugar chain that comprises a fucose residue at residue N297 according to the EU numbering. In some embodiments, at least one of the one or more analytic peptides comprises at least one amino acid residue from a CDR of the anti-CD40 antibody or the antigen-binding fragment thereof.

In some embodiments, at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11). In some embodiments, at least one of the one or more analytic peptides comprises the amino acid sequence of LLIYTVSNR (SEQ ID NO: 10).

In some embodiments, the sample is plasma or serum. In some embodiments, the plasma sample is treated with an anticoagulant. In some embodiments, the anticoagulant is dipotassium ethylenediaminetetraacetic acid (K2EDTA).

In some embodiments, the sample is from a human patient that has been administered the anti-CD40 antibody or antigen-binding fragment thereof. In some embodiments, the sample is at least 25, 50, 75, 100, or 200 μL. In some embodiments, the sample is about 25, about 50, about 75, about 100, or about 200 μL. In some embodiments, the sample is 25, 50, 75, 100, or 200 μL. In some embodiments, the sample is between 25-50, 50-75, 75-100, 100-125, 125-150, 150-175, or 175-200 μL.

In some embodiments, the method has a lower limit of quantification (LLOQ) of about 0.1 ng/mL to about 1 ng/ml. In some embodiments, the method has a lower limit of quantification (LLOQ) of about 0.5 ng/mL.

In some embodiments, the one or more analytic peptides are detected by a liquid chromatography tandem mass spectrometry (LC-MS/MS) system. In some embodiments, the LC-MS/MS system comprises a trap column and a nano column. In some embodiments, the trap column and the nano column are run at a temperature between about 50° C. to about 70° C. In some embodiments, nano-electrospray ionization with high resolution (LC-HRMS/MS) is used in the LC-MS/MS system. In some embodiments, the LC-HRMS/MS system is in the positive ion mode.

In some embodiments, the anti-CD40 antibody or antigen-binding fragment thereof has a concentration of about 0.5 ng/mL to about 50.0 ng/mL in the sample.

In some embodiments, an isotope-labeled peptide is added to the sample before digesting the extracted anti-CD40 antibody or antigen-binding fragment thereof. In some embodiments, the isotope-labeled peptide is from an isotope-labeled anti-CD40 antibody or antigen-binding fragment thereof. In some embodiments, the isotope-labeled peptide comprises PGKAPKLLIYTV\hat{ }SNR\hat{ }FSGVPS (SEQ ID NO: 12). In some embodiments, V\hat{ } represents ¹³C and/or ¹⁵N labeled valine, and R\hat{ } represents ¹³C and/or ¹⁵N labeled arginine.

In one aspect, the disclosure provides a method for determining the amount of an anti-CD40 antibody or antigen-binding fragment thereof in a sample. In some embodiments, the anti-CD40 antibody or antigen-binding fragment thereof comprises: a heavy chain variable region (VH) comprising an amino acid sequence of SEQ ID NO: 13; and a light chain variable region (VL) comprising an amino acid sequence of SEQ ID NO: 14. In some embodiments, the method comprises extracting the anti-CD40 antibody or antigen-binding fragment thereof from the sample; mixing a fixed amount of an isotope-labeled peptide with the extracted anti-CD40 antibody or antigen-binding fragment thereof; digesting the extracted anti-CD40 antibody or antigen-binding fragment thereof and the isotope-labeled peptide with a protease to release one or more analytic peptides from the anti-CD40 antibody or antigen-binding fragment thereof and one or more isotope-labeled analytic peptide fragments from the isotope-labeled peptide; and detecting the one or more analytic peptides and the one or more isotope-labeled analytic peptide fragments by mass spectrometry, thereby determining the amount of the anti-CD40 antibody or antigen-binding fragment thereof in the sample.

In some embodiments, the isotope-labeled peptide is an isotope-labeled anti-CD40 antibody or antigen-binding fragment thereof In some embodiments, the isotope-labeled peptide comprises PGKAPKLLIYTV\hat{ }SNR\hat{ }FSGVPS (SEQ ID NO: 12). In some embodiments, V\hat{ } represents D C and/or ¹⁵N labeled valine, and R\hat{ } represents ¹³C and/or ¹⁵N labeled arginine.

In some embodiments, the anti-CD40 antibody or antigen-binding fragment thereof is extracted by anti-idiotypic affinity capture. In some embodiments, the extracted anti-CD40 antibody or antigen-binding fragment thereof is digested by either one or both of trypsin and Lys-C.

In some embodiments, the anti-CD40 antibody comprises a human constant region. In some embodiments, the anti-CD40 antibody is non-fucosylated. In some embodiments, the anti-CD40 antibody is SEA-CD40.

In some embodiments, less than 5% of the anti-CD40 antibody in the sample has an N-glycoside-linked sugar chain that comprises a fucose residue at residue N297 according to the EU numbering.

In some embodiments, at least one of the one or more analytic peptides comprises at least one amino acid residue from a CDR of the anti-CD40 antibody or the antigen-binding fragment thereof. In some embodiments, at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11). In some embodiments, at least one of the one or more analytic peptides comprises the amino acid sequence of LLIYTVSNR (SEQ ID NO: 10).

In some embodiments, the method has a lower limit of quantification (LLOQ) of about 0.1 ng/mL to 1 ng/ml. In some embodiments, the method has a lower limit of quantification (LLOQ) of about 0.5 ng/mL.

In some embodiments, the one or more analytic peptides are detected by a liquid chromatography tandem mass spectrometry (LC-MS/MS).

In one aspect, the disclosure provides a method for determining the amount of an anti-CD40 antibody or antigen-binding fragment thereof in a sample. In some embodiments, the anti-CD40 antibody or antigen-binding fragment thereof comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2. In some embodiments, the method comprises adding a fixed amount of an isotope-labeled antibody or antigen-binding fragment thereof to the sample, wherein the isotope-labeled antibody or antigen-binding fragment thereof comprises VH CDR1, VH CDR2, VH CDR3 of SEQ ID NO: 13 and VL CDR1, VL CDR2, VL CDR3 of SEQ ID NO: 14; extracting the anti-CD40 antibody or antigen-binding fragment thereof and the isotope-labeled antibody or antigen-binding fragment thereof from the sample; digesting the extracted anti-CD40 antibody or antigen-binding fragment thereof and the extracted isotope-labeled antibody or antigen-binding fragment thereof with a protease to release one or more analytic peptides from the anti-CD40 antibody or antigen-binding fragment thereof and one or more isotope-labeled peptides from the isotope-labeled antibody or antigen-binding fragment thereof; and detecting the one or more analytic peptides and the one or more isotope-labeled peptides by mass spectrometry, thereby determining the amount of the anti-CD40 antibody or antigen-binding fragment thereof in the sample.

In some embodiments, the isotope-labeled antibody or antigen-binding fragment thereof comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2.

In some embodiments, the isotope-labeled antibody or antigen-binding fragment thereof comprises a heavy chain variable region comprising amino acids 1-113 of SEQ ID NO: 1 and a light chain variable region comprising amino acids 1-113 of SEQ ID NO: 2.

In some embodiments, the isotope-labeled antibody comprises a human constant region. In some embodiments, the isotope-labeled antibody is non-fucosylated. In some embodiments, the isotope-labeled antibody is an isotope-labeled SEA-CD40. In some embodiments, less than 5% of isotope-labeled antibody in the fixed amount of an isotope-labeled antibody or antigen-binding fragment thereof has an N-glycoside-linked sugar chain that comprises a fucose residue at residue N297 according to the EU numbering.

In some embodiments, at least one of the one or more isotope-labeled peptides comprises at least one amino acid residue from a CDR of the isotope-labeled antibody or antigen-binding fragment thereof.

In some embodiments, at least one of the one or more isotope-labeled peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11). In some embodiments, at least one of the one or more isotope-labeled peptides comprises the amino acid sequence of LLIYTVSNR (SEQ ID NO: 10).

In some embodiments, the anti-CD40 antibody or antigen-binding fragment thereof and the isotope-labeled antibody or antigen-binding fragment thereof are extracted by anti-idiotypic affinity capture. In some embodiments, the extracted anti-CD40 antibody or antigen-binding fragment thereof and the isotope-labeled antibody or antigen-binding fragment thereof are digested by either one or both of trypsin and Lys-C. In some embodiments, the anti-CD40 antibody comprises a human constant region. In some embodiments, the anti-CD40 antibody is non- fucosylated. In some embodiments, the anti-CD40 antibody is SEA-CD40. In some embodiments, less than 5% of the anti-CD40 antibody or antigen-binding fragment thereof in the sample has an N-glycoside-linked sugar chain that comprises a fucose residue at residue N297 according to the EU numbering.

In some embodiments, at least one of the one or more analytic peptides comprises at least one amino acid residue from a CDR of the anti-CD40 antibody or the antigen-binding fragment thereof. In some embodiments, at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11). In some embodiments, at least one of the one or more analytic peptides comprises the amino acid sequence of LLIYTVSNR (SEQ ID NO: 10).

In some embodiments, the method has a lower limit of quantification (LLOQ) of about 0.1 ng/mL to 1 ng/ml. In some embodiments, the method has a lower limit of quantification (LLOQ) of about 0.5 ng/mL.

In some embodiments, the one or more analytic peptides are detected by a liquid chromatography tandem mass spectrometry (LC-MS/MS).

In one aspect, the disclosure provides a method for determining the amount of an anti-CD40 antibody in a plasma sample, wherein the anti-CD40 antibody comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2; and a human constant region; the method comprising a) extracting the anti-CD40 antibody from the sample by anti-idiotypic affinity capture, wherein the plasma sample is obtained from a subject after being administered with a composition comprising the anti-CD40 antibody, wherein less than 5% of the anti-CD40 antibody in the composition has an N-glycoside-linked sugar chain that comprises a fucose residue at residue N297 according to the EU numbering; b) digesting the extracted anti-CD40 antibody with one or both of trypsin and Lys-C to release one or more analytic peptides from the anti-CD40 antibody; and c) detecting the one or more analytic peptides by a nanoscale liquid chromatography tandem mass spectrometry (nano LC-MS/MS), thereby determining the amount of the anti-CD40 antibody in the plasma sample.

In some embodiments, the method further comprises adding a fixed amount of an isotope-labeled peptide to the sample before digesting the extracted anti-CD40 antibody.

In some embodiments, at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11). In some embodiments, at least one of the one or more analytic peptides comprises the amino acid sequence of LLIYTVSNR (SEQ ID NO: 10).

In some embodiments, the method has a lower limit of quantification (LLOQ) of about 0.1 ng/mL to 1 ng/ml. In some embodiments, the method has a lower limit of quantification (LLOQ) of about 0.5 ng/mL.

In one aspect, the disclosure provides a peptide or peptide fragment comprising an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), NTAYLQMNSLR (SEQ ID NO: 7), EGIYWWGQGTLVTVSSASTK (SEQ ID NO: 8), SSQSLVHSNGNTFLHWYQQKPGK (SEQ ID NO: 9), LLIYTVSNR (SEQ ID NO: 10), and FSGVPSR (SEQ ID NO: 11). In some embodiments, the peptide or peptide fragment comprises the amino acid sequence LLIYTVSNR (SEQ ID NO: 10). In some embodiments, the peptide or peptide fragment is used in a method for determining the amount of an anti-CD40 antibody in a sample.

In one aspect, the disclosure provides a peptide or peptide fragment comprising the amino acid sequence PGKAPKLLIYTVSNRFSGVPS (SEQ ID NO: 12).

In some embodiments, the peptide or peptide fragment comprises at least one isotopic label. In some embodiments, the at least one isotopic label is selected from D C and ¹⁵N.

In some embodiments, the peptide or peptide fragment comprises PGKAPKLLIYTV\hat{ }SNR\hat{ }FSGVPS. In some embodiments, V\hat{ } represents ¹³C and/or ¹⁵N labeled valine, and R\hat{ } represents ¹³C and/or ¹⁵N labeled arginine.

In one aspect, the disclosure provides a method for determining the pharmacokinetics of an anti-CD40 antibody or antigen-binding fragment thereof in a subject, wherein the anti-CD40 antibody or antigen-binding fragment thereof comprises: a heavy chain variable region (VH) comprising an amino acid sequence of SEQ ID NO: 13; and a light chain variable region (VL) comprising an amino acid sequence of SEQ ID NO: 14; the method comprising obtaining two or more samples from the subject at different time points after administering a composition comprising the anti-CD40 antibody or antigen-binding fragment thereof to the subject; extracting the anti-CD40 antibody or antigen-binding fragment thereof from each of the two or more samples; digesting the extracted anti-CD40 antibody or antigen-binding fragment thereof from each of the two or more samples separately with a protease to release one or more analytic peptides from the anti-CD40 antibody or antigen-binding fragment thereof in each of the two or more samples; and detecting the one or more analytic peptides derived from each of the two or more samples by mass spectrometry, thereby determining the pharmacokinetics of the anti-CD40 antibody or antigen-binding fragment thereof in the subject.

In some embodiments, the anti-CD40 antibody or antigen-binding fragment thereof is extracted by anti-idiotypic affinity capture. In some embodiments, the extracted anti-CD40 antibody or antigen-binding fragment thereof is digested by either one or both of trypsin and Lys-C. In some embodiments, the anti-CD40 antibody comprises a human constant region. In some embodiments, the anti-CD40 antibody is non-fucosylated. In some embodiments, the anti-CD40 antibody is SEA-CD40. In some embodiments, less than 5% of the anti-CD40 antibody or antigen-binding fragment thereof in the composition has an N-glycoside-linked sugar chain that comprises a fucose residue at residue N297 according to the EU numbering. In some embodiments, at least one of the one or more analytic peptides comprises at least one amino acid residue from a CDR of the anti-CD40 antibody or the antigen-binding fragment thereof

In some embodiments, at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11). In some embodiments, at least one of the one or more analytic peptides comprises the amino acid sequence of LLIYTVSNR (SEQ ID NO: 10).

In some embodiments, the sample is plasma. In some embodiments, the method has a lower limit of quantification (LLOQ) of about 0.1 ng/mL to about 1 ng/ml. In some embodiments, the method has a lower limit of quantification (LLOQ) of about 0.5 ng/mL.

In some embodiments, the one or more analytic peptides are detected by a liquid chromatography tandem mass spectrometry (LC-MS/MS) system. In some embodiments, nano-electrospray ionization with high resolution (LC-HRMS/MS) is used in the LC-MS/MS system.

In some embodiments, the anti-CD40 antibody or antigen-binding fragment thereof has a concentration of about 0.5 ng/mL to about 50.0 ng/mL in in each of the two or more samples.

In some embodiments, an isotope-labeled peptide is added to the sample before digesting the extracted anti-CD40 antibody or antigen-binding fragment thereof. In some embodiments, the isotope-labeled peptide is an isotope-labeled anti-CD40 antibody or antigen-binding fragment thereof. In some embodiments, the isotope-labeled peptide comprises PGKAPKLLIYTV\hat{ }SNR\hat{ }FSGVPS (SEQ ID NO: 12). In some embodiments, V\hat{ } represents ¹³C and/or ¹⁵N labeled valine, and R\hat{ } represents ¹³C and/or ¹⁵N labeled arginine.

In one aspect, the disclosure provides a method for monitoring the amount of an anti-CD40 antibody or antigen-binding fragment thereof in a subject. In some embodiments, the anti-CD40 antibody or antigen-binding fragment thereof comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2. In some embodiments, the method comprises obtaining a first sample from the subject at a first time point after administering a composition comprising the anti-CD40 antibody or antigen-binding fragment thereof to the subject; extracting the anti-CD40 antibody or antigen-binding fragment thereof from the first sample; digesting the extracted anti-CD40 antibody or antigen-binding fragment thereof from the first sample with a protease to release one or more analytic peptides from the anti-CD40 antibody or antigen-binding fragment thereof; and detecting the one or more analytic peptides by mass spectrometry, thereby determining the amount of the anti-CD40 antibody or antigen-binding fragment thereof in the first sample.

In some embodiments, the method further comprises obtaining a second sample from the subject at a second time point that is different from the first time point from the subject after administering the composition comprising the anti-CD40 antibody or antigen-binding fragment thereof to the subject; extracting the anti-CD40 antibody or antigen-binding fragment thereof from the second sample; digesting the extracted anti-CD40 antibody or antigen-binding fragment thereof from the second sample with a protease to release one or more analytic peptides from the anti-CD40 antibody or antigen-binding fragment thereof; detecting the one or more analytic peptides by mass spectrometry, thereby determining the amount of the anti-CD40 antibody or antigen-binding fragment thereof in the second sample.

In some embodiments, the method further comprises comparing the amount of the one or more analytic peptides in the first and second samples to monitor the amount of the anti-CD40 antibody or antigen-binding fragment thereof in the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIGS. 1A and 1B provide the heavy and light chain amino acid sequences for the SEA-CD40 antibody. FIG. 1B indicates Lysine (K) and Arginine (R) trypsin cleavage sites in bold type font, CDR regions are underlined, and signature peptides are highlighted by gray color.

FIG. 2 illustrates an embodiment of a method described herein. An antibody of interest is extracted using α-ID affinity capture and is digested with trypsin. The digested peptides are loaded into a 2D chromatography system, which may contain a loading column and a nano flow column. The resulting peptides are then measured using nano tandem mass spectrometry (MS/MS).

FIG. 3 provides a representative full-scan nano-electrospray mass spectrum of SEA-CD40 L peptide.

FIG. 4 provides a representative full-scan higher-energy collisional dissociation (HCD) product ion mass spectrum of SEA-CD40 L peptide.

FIG. 5 provides a full-scan nano-electrospray mass spectrum of SEA-CD40 internal standard (IS).

FIG. 6 provides a representative full-scan HCD product ion mass spectrum of SEA-CD40 IS.

FIGS. 7A and 7B provide extracted accurate mass chromatograms of SEA-CD40 L peptide (FIG. 7A) and SEA-CD40 IS (FIG. 7B) from a calibration standard human plasma extract (0.500 ng/mL).

FIGS. 8A-8C provide chromatograms for the control blank (FIG. 8A), zero blank (FIG. 8B) and 0.50 ng/mL SEA-CD40 standard (FIG. 8C) for a trypsin/Lys-C digestion.

FIG. 9A shows SEA-CD40 plasma concentration versus time profiles for SEA-CD40 following IV administration Q3wk in participants with solid tumor malignancies. LLOQ is the lower limit of quantitation.

FIG. 9B shows SEA-CD40 plasma concentration versus time profiles for SEA-CD40 following IV administration Q3wk in participants with lymphomas.

FIG. 9C shows SEA-CD40 plasma concentration versus time profiles for SEA-CD40 following IV administration Q3wk in participants with solid tumor malignancies after Cycle 1.

FIG. 9D shows SEA-CD40 plasma concentration versus time profiles for SEA-CD40 following IV administration Q3wk in participants with lymphomas after Cycle 1.

FIG. 10 shows SEA-CD40 PK parameter summaries for dose escalation cohorts in participants with solid tumors and lymphomas.

FIG. 11A shows the results from calibration curve using SILAC.

FIG. 11B shows the results from calibration curve using IS.

FIG. 12A shows the results from quality control analysis of SILAC.

FIG. 12B shows the results from quality control analysis using IS.

FIG. 13 provides several sequences as described in the present disclosure.

### DETAILED DESCRIPTION

Antibodies can readily be detected and quantified with a variety of immunological techniques known in the art, such as the use of enzyme-linked immunosorbent assay (ELISA), immunoprecipitation, fluorescence activated cell sorting (FACS), etc. (See, e.g., Ausubel et al., eds., Short Protocols in Molecular Biology (John Wiley and Sons, Inc., New York, 4th ed. 1999); Harlow and Lane, Using Antibodies: A Laboratory Manual (Cold Spring Harbor Laboratory Press, Cold Spring Harbor, N.Y., 1999).

Initial efforts to quantify anti-CD40 antibodies in human subject samples utilized an ELISA assay. ELISAs typically comprise coating the well of a 96 well microtiter plate with a prepared antigen (e.g., CD40), adding the antibody conjugated to a detectable compound such as an enzyme (e.g., horseradish peroxidase or alkaline phosphatase) to the well and incubating for a period of time, and detecting the presence of the antibody. In ELISAs, the antibody does not have to be conjugated to a detectable compound; instead, a second antibody (i.e. an anti-idiotypic antibody (α-ID) which recognizes the antibody of interest) conjugated to a detectable compound may be added to the well. Further, instead of coating the well with the antigen, the antibody may be coated to the well. In this case, a second antibody conjugated to a detectable compound may be added following the addition of protein (e.g., CD40) to the coated well. For further discussion regarding ELISAs, see, for example, Ausubel et al., eds., 1994, Current Protocols in Molecular Biology, Vol. 1, John Wiley & Sons, Inc., New York at 11.2.1.

For the anti-CD40 antibody SEA-CD40, an ELISA was performed utilizing α-ID affinity capture and detection via a selected labeled α-ID. As shown in Table 1, the majority of non-EOI (end of infusion) samples taken from patients had a signal that was below the limit of quantification (BLQ) (<15 ng/mL) for ELISA. Accordingly, a method with improved sensitivity was required for accurately quantifying anti-CD40 antibodies in a sample.

The present disclosure provides methods for determining the amount of an anti-CD40 antibody in a sample. In one aspect, the method utilizes a hybrid approach of immunocapture, trypsin digestion, and quantification with LC-MS/MS to determine the amount of an anti-CD40 antibody in a sample (see FIG. 2). The methods of the present disclosure help to eliminate the selectivity and sensitivity limitations of prior anti-CD40 antibody quantification methods. Particularly, the methods described herein can greatly improve the sensitivity of the detection, with the lowest level of quantitation concentration of e.g., about 0.5 ng/mL.

TABLE 1

| | | | Hr. | |
|---|---|---|---|---|
| Dose | Subject | Cycle | Post Dose | SEA-CD40, ng/mL |
| 30 | Subject 1 | 1 | 0 | BLQ(<15 ng/mL) |
| | | | EOI | 19.4 |
| | | | 2 | BLQ(<15 ng/mL) |
| | | | 4 | BLQ(<15 ng/mL) |
| | | | 24 | BLQ(<15 ng/mL) |
| | | | 72 | BLQ(<15 ng/mL) |
| | | | 168 | BLQ(<15 ng/mL) |
| | | | 336 | BLQ(<15 ng/mL) |
| | | 2 | 0 | BLQ(<15 ng/mL) |
| | | | EOI | 35.4 |
| | | | 2 | BLQ(<15 ng/mL) |
| | | | 4 | BLQ(<15 ng/mL) |
| | | | 24 | BLQ(<15 ng/mL) |
| | | | 72 | BLQ( <15 ng/mL) |
| | | | 168 | BLQ(<15 ng/mL) |
| | | | 336 | BLQ(<15 ng/mL) |
| 30 | Subject 2 | 1 | 0 | BLQ(<15 ng/mL) |
| | | | EOI | 42.9 |
| | | | 2 | BLQ(<15 ng/mL) |
| | | | 4 | BLQ(<15 ng/mL) |
| | | | 24 | BLQ(<15 ng/mL) |
| | | | 72 | BLQ(<15 ng/mL) |
| | | | 168 | BLQ(<15 ng/mL) |
| | | | 336 | BLQ(<15 ng/mL) |
| | | 2 | 0 | BLQ(<15 ng/mL) |
| | | | EOI | BLQ(<15 ng/mL) |
| | | | 2 | BLQ(<15 ng/mL) |
| | | | 4 | BLQ(<15 ng/mL) |
| | | | 24 | BLQ(<15 ng/mL) |
| | | | 168 | BLQ(<15 ng/mL) |
| | | | 336 | BLQ(<15 ng/mL) |

### Definitions

A “polypeptide” or “polypeptide chain” is a polymer of amino acid residues joined by peptide bonds, whether produced naturally or synthetically, and does not refer to a specific length; thus, “peptides” and “proteins” are included within the definition of a polypeptide. Also included within the definition of proteins are “antibodies” as defined herein. A “polypeptide region” refers to a segment of a polypeptide, which segment can contain, for example, one or more domains or motifs (e.g., a polypeptide region of an antibody can contain, for example, one or more CDRs).

As used herein, the term “fragment” or “peptide fragment” refers to a portion of a polypeptide typically having at least 5, 10, 20, 30, 40, or 50 contiguous amino acids of the polypeptide.

A “protein” is a macromolecule comprising one or more polypeptide chains. A protein can also comprise non-peptidic components, such as carbohydrate groups. Carbohydrates and other non-peptidic substituents can be added to a protein by the cell in which the protein is produced, and will vary with the type of cell. Proteins are defined herein in terms of their amino acid backbone structures; substituents such as carbohydrate groups are generally not specified, but can be present nonetheless.

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “antibody” denotes immunoglobulin proteins produced in response to the presence of an antigen and that bind to the antigen, as well as antigen-binding fragments and engineered variants thereof. Hence, the term “antibody” includes, for example, intact monoclonal antibodies comprising full-length immunoglobulin heavy and light chains (e.g., antibodies produced using hybridoma technology) and antigen-binding antibody fragments, such as F(ab')2 and Fab fragments. Genetically engineered intact antibodies and fragments, such as chimeric antibodies, humanized antibodies, single-chain Fv fragments, single-chain antibodies, diabodies, minibodies, linear antibodies, multivalent or multispecific (e.g., bispecific) hybrid antibodies, and the like are also included. Thus, the term “antibody” is used expansively to include any protein, derived from any species (e.g. human, primate, mouse, etc.), that comprises an antigen-binding site of an antibody and is capable of specifically binding to its antigen.

An “antigen-binding site of an antibody” is that portion of an antibody that is sufficient to bind to its antigen. At a minimum, the antigen-binding site of an antibody is a variable domain or a genetically engineered variant thereof. Single-domain binding sites can be generated from camelid antibodies (see Muyldermans and Lauwereys, J. Mol. Recog. 12:131-140, 1999; Nguyen et al., EMBO J. 19:921-930, 2000) or from VH domains of other species to produce single-domain antibodies (“dAbs”; see Ward et al., Nature 341:544-546, 1989; U.S. Pat. No. 6,248,516 to Winter et al.). In certain variations, an antigen-binding site is a polypeptide region having only two complementarity determining regions (CDRs) of a naturally or non-naturally (e.g., mutagenized) occurring heavy chain variable domain or light chain variable domain, or combination thereof (see, e.g., Pessi et al., Nature 362:367-369, 1993; Qiu et al., Nature Biotechnol. 25:921-929, 2007). More commonly, an antigen-binding site of an antibody comprises both a heavy chain variable (VH) domain and a light chain variable (VL) domain that bind to an epitope. In some embodiments, an antibody can include one or more components in addition to an antigen-binding site, such as, for example, a second antigen-binding site of an antibody (which can bind to the same or a different epitope or to the same or a different antigen), a peptide linker, an immunoglobulin constant region, an immunoglobulin hinge, an amphipathic helix (see Pack and Pluckthun, Biochem. 31:1579-1584, 1992), a non-peptide linker, an oligonucleotide (see Chaudri et al., FEBS Letters 450:23-26, 1999), a cytostatic or cytotoxic drug, and the like, and can be a monomeric or multimeric protein. Examples of molecules comprising an antigen-binding site of an antibody are known in the art and include, for example, Fv, single-chain Fv (scFv), Fab, Fab′, F(ab′)₂, F(ab)_{c}, diabodies, dAbs, minibodies, nanobodies, Fab-scFv fusions, bispecific (scFv)₄-IgG, and bispecific (scFv)₂-Fab (see, e.g., Hu et al., Cancer Res. 56:3055-3061, 1996; Atwell et al., Molecular Immunology 33:1301-1312, 1996; Carter and Merchant, Curr. Opin. Biotechnol. 8:449-454, 1997; Zuo et al., Protein Engineering 13:361-367, 2000; and Lu et al., J. Immunol. Methods 267:213-226, 2002).

The term “immunoglobulin” refers to a protein consisting of one or more polypeptides substantially encoded by immunoglobulin gene(s). One form of immunoglobulin constitutes the basic structural unit of native (i.e., natural) antibodies in vertebrates. This form is a tetramer and consists of two identical pairs of immunoglobulin chains, each pair having one light chain and one heavy chain. In each pair, the light and heavy chain variable regions (VL and VH) are together primarily responsible for binding to an antigen, and the constant regions are primarily responsible for the antibody effector functions. Five classes of immunoglobulin proteins (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a yl heavy chain constant region. Each immunoglobulin heavy chain possesses a constant region that consists of constant region protein domains (CH1, hinge, CH2, and CH3; IgG3 also contains a CH4 domain) that are essentially invariant for a given subclass in a species. DNA sequences encoding human and non-human immunoglobulin chains are known in the art (see, e.g., Ellison et al., DNA 1:11-18, 1981; Ellison et al.,Nucleic Acids Res. 10:4071-4079, 1982; Kenten et al., Proc. Natl. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228). For a review of immunoglobulin structure and function, see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

Full-length immunoglobulin “light chains” (about 25 Kd or 214 amino acids) are encoded by a variable region gene at the amino-terminus (encoding about 110 amino acids) and a by a kappa or lambda constant region gene at the carboxyl-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally, Fundamental Immunology, 2nd ed., W.E. Paul (Ed.), Raven Press, N.Y., 1989, Ch. 7).

An immunoglobulin light or heavy chain variable region (also referred to herein as a “light chain variable domain” (“VL domain”) or “heavy chain variable domain” (“VH domain”), respectively) consists of a “framework” region interrupted by three hypervariable regions, also called “complementarity determining regions” or “CDRs.” The framework regions serve to align the CDRs for specific binding to an epitope of an antigen. Thus, the term “hypervariable region” or “CDR” refers to the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, FR4.

The assignment of amino acids to each domain is in accordance with the definitions of Kabat, Sequences of Proteins of Immunological Interest (National Institutes of Health, Bethesda, MD, 1987 and 1991), or Chothia & Lesk, J. Mol. Biol. 196:901-917, 1987; Chothia et al., Nature 342:878-883, 1989. Kabat also provides a widely used numbering convention (Kabat numbering) in which corresponding residues between different heavy chains or between different light chains are assigned the same number. CDRs 1, 2, and 3 of a VL domain are also referred to herein, respectively, as CDR-L1, CDR-L2, and CDR-L3; CDRs 1, 2, and 3 of a VH domain are also referred to herein, respectively, as CDR-H1, CDR-H2, and CDR-H3.

The term “genetically engineered antibody” refers to an antibody in which the amino acid sequence has been varied from that of the native or parental antibody. The possible variations are many, and range from the changing of just one or a few amino acids to the complete redesign of, for example, the variable or constant region. Changes in the constant region are, in general, made to improve or alter characteristics such as, complement binding and other effector functions. Typically, changes in the variable region are made to improve antigen-binding characteristics, improve variable region stability, and/or reduce the risk of immunogenicity.

The term “monoclonal antibody” or “mAbs” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. Unless the context dictates otherwise, the term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. The monoclonal antibodies herein also specifically include “chimeric” antibodies.

The term “chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is (are) identical with or homologous to corresponding sequences in an antibody derived from another species (e.g., mouse) or belonging to another antibody class or subclass, as well as fragments of such antibodies, so long as they exhibit the desired biological activity. Chimeric immunoglobulins or antibodies can be constructed, for example by genetic engineering, from immunoglobulin gene segments belonging to different species. The term “humanized antibody,” as defined infra, is not intended to encompass chimeric antibodies. Although humanized antibodies are chimeric in their construction (i.e., comprise regions from more than one species of protein), they include additional features (i.e., variable regions comprising donor CDR residues and acceptor framework residues) not found in chimeric immunoglobulins or antibodies, as defined herein.

The term “humanized VH domain” or “humanized VL domain” refers to an immunoglobulin VH or VL domain comprising some or all CDRs entirely or substantially from a non-human donor immunoglobulin (e.g., a mouse or rat) and variable region framework sequences entirely or substantially from human immunoglobulin sequences. The non-human immunoglobulin providing the CDRs is called the “donor” and the human immunoglobulin providing the framework is called the “acceptor.” In some instances, humanized antibodies can retain non-human residues within the human variable domain framework regions to enhance proper binding characteristics (e.g., mutations in the frameworks can be required to preserve binding affinity when an antibody is humanized).

A “humanized antibody” is an antibody comprising one or both of a humanized VH domain and a humanized VL domain. Immunoglobulin constant region(s) need not be present, but if they are, they are entirely or substantially from human immunoglobulin constant regions.

In a humanized antibody, the CDRs are from a non-human “donor” antibody and are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557). The acceptor antibody sequences can be, for example, a mature human antibody sequence, a composite of such sequences, a consensus sequence of human antibody sequences, or a germline region sequence. Human acceptor sequences can be selected for a high degree of sequence identity in the variable region frameworks with donor sequences to match canonical forms between acceptor and donor CDRs among other criteria. Thus, a humanized antibody is an antibody having CDRs entirely or substantially from a donor antibody, and variable region framework sequences and constant regions, if present, entirely or substantially from human antibody sequences. Similarly, a humanized heavy chain typically has all three CDRs entirely or substantially from a donor antibody heavy chain, and a heavy chain variable region framework sequence and heavy chain constant region, if present, substantially from human heavy chain variable region framework and constant region sequences. Similarly, a humanized light chain typically has all three CDRs entirely or substantially from a donor antibody light chain, and a light chain variable region framework sequence and light chain constant region, if present, substantially from human light chain variable region framework and constant region sequences.

A CDR in a humanized antibody is “substantially from” a corresponding CDR in a non-human antibody when at least 60%, at least 85%, at least 90%, at least 95% or 100% of corresponding residues (as defined by Kabat (or IMGT)) are identical between the respective CDRs; or when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering), or wherein about 100% of corresponding residues (as defined by Kabat numbering), are identical between the respective CDRs. The variable region framework sequences of an antibody chain or the constant region of an antibody chain are substantially from a human variable region framework sequence or human constant region respectively when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical.

Although humanized antibodies often incorporate all six CDRs (preferably as defined by Kabat) from a mouse antibody, they can also be made with fewer than all six CDRs (e.g., at least 3, 4, or 5) from a mouse antibody (see, e.g., Pascalis et al., J. Immunol. 169:3076, 2002; Vaj dos et al., J. Mol. Biol., 320: 415-428, 2002; Iwahashi et al., Mol. Immunol., 36:1079-1091, 1999; Tamura et al., J. Immunol., 164: 1432- 1441, 2000).

In particular variations of a humanized VH or VL domain in which CDRs are substantially from a non-human immunoglobulin, the CDRs of the humanized VH or VL domain have no more than six (e.g., no more than five, no more than four, no more than three, no more than two, or nor more than one) amino acid substitutions (preferably conservative substitutions) across all three CDRs relative to the corresponding non-human VH or VL CDRs. The variable region framework sequences of an antibody VH or VL domain or, if present, a sequence of an immunoglobulin constant region are “substantially from” a human VH or VL framework sequence or human constant region, respectively, when at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% of corresponding residues (as defined by Kabat numbering for the variable region and EU numbering for the constant region), or about 100% of corresponding residues (as defined by Kabat numbering for the variable region and EU numbering for the constant region) are identical. Hence, all parts of a humanized antibody, except the CDRs, are typically entirely or substantially from corresponding parts of natural human immunoglobulin sequences.

Specific binding of an antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not, however, necessarily imply that a monoclonal antibody binds one and only one target.

The term “epitope” refers to a site of an antigen to which an antibody binds. An epitope can be formed from contiguous amino acids or noncontiguous amino acids juxtaposed by tertiary folding of one or more proteins. Epitopes formed from contiguous amino acids are typically retained upon exposure to denaturing agents, e.g., solvents, whereas epitopes formed by tertiary folding are typically lost upon treatment with denaturing agents, e.g., solvents. An epitope typically includes at least about 3, and more usually, at least about 5, at least about 6, at least about 7, or about 8-10 amino acids in a unique spatial conformation. Methods of determining spatial conformation of epitopes include, for example, x-ray crystallography and two-dimensional nuclear magnetic resonance (see, e.g., Epitope Mapping Protocols in Methods in Molecular Biology, Vol. 66, Glenn E. Morris (Ed.) (1996)).

The intact antibody can have one or more “effector functions,” which refer to those biological activities attributable to the Fc region (a native sequence Fc region or amino acid sequence variant Fc region) of an antibody. Examples of antibody effector functions include, but are not limited to: C1q binding; complement dependent cytotoxicity (CDC); Fc receptor binding; antibody-dependent cell-mediated cytotoxicity (ADCC); phagocytosis; and down regulation of cell surface receptors (e.g., B cell receptor; BCR).

With regard to proteins as described herein, reference to amino acid residues corresponding to those specified by SEQ ID NO includes post-translational modifications of such residues.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison, Wisconsin). Other methods for comparing two nucleotide or amino acid sequences by determining optimal alignment are well-known to those of skill in the art (see, e.g., Peruski and Peruski, The Internet and the New Biology: Tools for Genomic and Molecular Research (ASM Press, Inc. 1997); Wu et al. (Eds.), “Information Superhighway and Computer Databases of Nucleic Acids and Proteins,” in Methods in Gene Biotechnology 123-151 (CRC Press, Inc. 1997); Bishop (Ed.), Guide to Human Genome Computing (2nd ed., Academic Press, Inc. 1998)). Two amino acid sequences are considered to have “substantial sequence identity” if the two sequences have at least about 80%, at least about 85%, at least about 90%, or at least about 95% sequence identity relative to each other.

Percentage sequence identities are determined with antibody sequences maximally aligned by the Kabat numbering convention. After alignment, if a subject antibody region (e.g., the entire variable domain of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

As used herein, a “complex N-glycoside-linked sugar chain” is typically bound to asparagine 297 (according to the EU index as set forth in Kabat, “Sequences of Immunological Interest, 5th Ed., Pub. No. 91-3242, U.S. Dept. Healtth & Human Services, NIH, Bethesda, MD, 1991). As used herein, the complex N-glycoside-linked sugar chain has a biantennary composite sugar chain, mainly having the following structure:

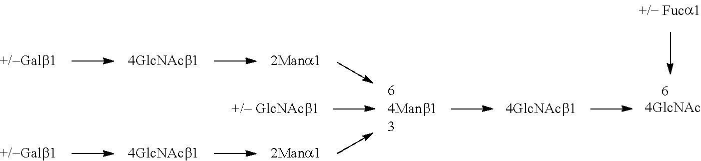

where +/− indicates the sugar molecule can be present or absent, and the numbers indicate the position of linkages between the sugar molecules. In the above structure, the sugar chain terminal, which binds to asparagine, is called a reducing terminal (at right), and the opposite side is called a non-reducing terminal. Fucose is usually bound to N-acetylglucosamine (“GlcNAc”) of the reducing terminal, typically by an α1,6 bond (the 6-position of GlcNAc is linked to the 1-position of fucose). “Gal” refers to galactose, and “Man” refers to mannose.

A “complex N-glycoside-linked sugar chain” includes 1) a complex type, in which the non-reducing terminal side of the core structure has one or more branches of galactose-N-acetylglucosamine (also referred to as “gal-GlcNAc”) and the non-reducing terminal side of Gal-GlcNAc optionally has a sialic acid, bisecting N-acetylglucosamine or the like; or 2) a hybrid type, in which the non-reducing terminal side of the core structure has both branches of a high mannose N-glycoside-linked sugar chain and complex N-glycoside-linked sugar chain.

An “analytic peptide” or “signature peptide” refers to peptide that is released or cleaved from an antibody, and which is detected or measured (quantitated) by one or more known analytic techniques, e.g. mass spectrometry. The analytic peptide can contain a CDR amino acid sequence or a portion thereof (i.e. at least one CDR amino acid). The amount of analytic peptide is representative of the amount of the antibody from which it is released or cleaved.

The terms “extract,” “extracted,” “extraction,” and “extracting” refer to removal of an antibody from a heterogeneous sample comprising several proteins and other molecules. Any appropriate method or material known in the art that can selectively extract an antibody from a heterogeneous sample, particularly a biological sample, can be employed in the methods herein. Extraction, for example, can include: affinity chromatography, size exclusion chromatography, ammonium sulfate precipitation, ion exchange chromatography, immobilized metal chelate chromatography, and immunoprecipitation. In a particular example, extraction can include immunoaffinity chromatography with anti-idiotypic antibodies, also referred herein as “anti-idiotypic affinity capture.”

An “anti-idiotypic antibody” (α-ID) refers to an antibody that binds to the antigen-binding site of another antibody. In some embodiments, the α-ID antibody can contain a label (e.g. biotin). An anti-idiotypic antibody can bind to the idiotype of another antibody. An idiotype can be defined as the specific combination of idiotopes present within an antibodies complement determining regions (CDRs).

As used herein, the terms “eluent” or “elution” refers to the liquid mobile phase that dissociates analytes from the chromatography column stationary phase. Analytes include, but are not limited to, compound(s) of interest to be measured.

The terms “release,” “released,” and “releasing” refer to extracellular cleavage of an analytic peptide from an antibody. For a given antibody, the amount of analytic peptide released will typically vary depending the amino acid sequence, the type of extraction and conditions thereof used to remove the antibody from a sample, treatment of the antibody after extraction, and method and conditions of the extracellular cleavage. For consistency of results from sample to sample, the same reaction conditions should be employed.

A “tryptic digest solution” refers to a solution containing trypsin. A tryptic digest solution can contain other components in addition to trypsin. For example, the tryptic digest solution can contain trypsin and Lys C. The solution can also contain a resuspension buffer or water for reconstitution or dilution. The conditions of the tryptic digest solution can vary depending on factors such as the amount of analyte, type of analyte, or the extraction procedure used to isolate an analyte from a sample.

A “cytotoxic effect” refers to the depletion, elimination and/or killing of a target cell. A “cytotoxic agent” refers to a compound that has a cytotoxic effect on a cell, thereby mediating depletion, elimination and/or killing of a target cell. The term includes radioactive isotopes (e.g., 211At, 131I, 125I, 90Y, 186Re, 188Re, 153 Sm, 212Bi, 32P, 60C, and radioactive isotopes of Lu), chemotherapeutic agents, and toxins such as small molecule toxins or enzymatically active toxins of bacterial, fungal, plant or animal origin, including synthetic analogs and derivatives thereof. In certain embodiments, a cytotoxic agent is conjugated to an antibody or administered in combination with an antibody.

A “cytostatic effect” refers to the inhibition of cell proliferation. A “cytostatic agent” refers to a compound that has a cytostatic effect on a cell, thereby mediating inhibition of growth and/or expansion of a specific cell type and/or subset of cells.

The term “patient” or “subject” includes human and non-human subjects, such as, but not limited to, primates, rabbits, rats, mice, etc. and transgenic species thereof, that receive either prophylactic or therapeutic treatment.

The terms “treat,” “treating,” “treated” or “treatment,” unless otherwise indicated by context, refer to therapeutic treatment and prophylactic measures to prevent relapse, wherein the object is to inhibit or slow down (lessen) an undesired physiological change or disorder. “Treatment” can also mean prolonging survival as compared to expected survival if not receiving treatment. Those in need of treatment include those known to have the condition or disorder, those suspected of having the condition or disorder, as well as those prone to having the condition or disorder.

The term “standard curve” or “calibration curve” refers to a graph used as a quantitative research technique. To generate the standard curve, multiple samples with known properties are measured and graphed, which then allows the same properties to be determined for unknown samples by interpolation on the graph. The samples with known properties are the standards, and the graph is the standard curve. Standard curves are of particular use when measuring the amount or concentration of an analyte in a sample that can contain an unknown amount of the analyte. The use of a standard curve alone represents the use of an external standard. As is understood in the art, the standard curve of a given analyte to be quantitated should generally span the concentration range of the analyte expected in the samples. Standard curve data can be processed to generate a function, e.g., a straight line (e.g., using linear regression analysis), which can be used for calculation of concentrations of unknowns. Again as is understood in the art, samples used for preparing the standard curve are processed by the same steps as test samples and any control samples in which the analyte is to be measured. A standard curve can also be employed in combination with the use of an internal standard. In this case, a constant (or fixed) amount of the internal standard is added to each sample used to generate the standard curve of known analyte concentrations. The same constant amount of internal standard is added to each test sample and to any blanks or control samples. The details of use of standard curves (calibration curves) as an external standard and a combination of the use of a standard curve with addition of internal standard for quantitation of analytes by analytic methods, including MS, LC-MS and LC-MS/MS methods, is well known in the art. One of ordinary skill in the art understands how to use such analytic methods in the determination of concentrations of analytes in a variety of samples, including biological samples as discussed herein.

An “internal standard” refers to a chemical species that behaves in a selected assay similarly to the chemical species to be quantitated (i.e., antibody or analytic peptide), but which is distinguishable from that chemical species in the analytic method being used. Typically, the internal standard is labeled to distinguish it from the chemical species to be quantitated, but the label employed does not significantly differentially affect its behavior compared to that of the chemical species to be quantitated. Preferably, anything that affects the measurement of the chemical species to be quantitated (e.g., analyte peak area) will also affect the measurement of the internal standard similarly. The ratio of the measurements of the chemical species to be quantitated and its internal standard preferably exhibits less variability than the measurement of the chemical species in a test sample. For use in mass spectrometry methods, the internal standard has a molecular weight that is different from the chemical species to be quantitated.

The terms “label,” “labeled,” and “labeling” refer to any moiety that can be covalently attached to polypeptide and that functions to: (i) provide a detectable signal; (ii) interact with a second label to modify the detectable signal provided by the first or second label, e.g. FRET (fluorescence resonance energy transfer); (iii) stabilize interactions or increase affinity of binding, with antigen or ligand; (iv) affect mobility, e.g. electrophoretic mobility, or cell-permeability, by charge, hydrophobicity, shape, or other physical parameters, or (v) provide a capture moiety, to modulate ligand affinity, antibody/antigen binding, or ionic complexation. Polypeptides can be conjugated with any label moiety which can be covalently attached to the polypeptide via a cysteine thiol. For diagnostic applications, the polypeptide will typically be labeled with a detectable moiety.

Most often labeling with stable isotopes, such as nitrogen 15 (¹⁵N) and carbon 13 (¹³C) is employed. Labeling must allow separate measurement of analyte and internal standard. Preferably, an isotopically labeled internal standard differs in molecular weight from the chemical species to be quantitated. Internal standards can also be surrogates of the chemical species to be quantitated. Surrogate internal standards differ structurally from the chemical species to be quantitated by substitution of an atom or chemical group by a different group, for example the substitution of a methyl group or other small alkyl for a hydrogen, or the substitution of a halogen, e.g., a fluorine, for a hydrogen. Such surrogates can be of particular use where it is not possible to readily obtain an isotopically labeled internal standard.

The terms “determine,” “determined,” and “determining” refer to the ascertaining of the concentration or amount of a particular antibody based on a measurement of the amount of an analytic peptide and the known amounts of one or more correlative factors. As is understood in the art, antibody concentration can be combined with the results of other measurements to determine other structural and physical properties of an antibody.

As used herein, the term “matrix” refers to the context or milieu in which a protein or protein agent conjugate compound is present. For example, a “matrix” includes formulation buffers, biological serum, surfactants, excipients or cell culture media.

A “surfactant” includes, but is not limited to, non-volatile compounds of the matrix. For example, surfactants include non-ionic and zwitterionic detergents, such as polysorbate 20 (“tween 20”) and polysorbate 80 (“tween 80”).

An “excipient” includes compounds such as sugars and polyols, for example, sucrose, trehalose, and sorbitol.

“Mass spectrometry” (MS) is an analytical technique used to measure the mass-to-charge ratio (m/z) of charged particles. The m/z is the ratio of an ion's mass (m) in atomic mass units (amu) to its formal charge (z). MS can be used for elucidating the primary structure of proteins. “Tandem mass spectrometry” (MS/MS) involves , fragmentation and detection of the fragment ions. “High resolution mass spectrometry” (HRMS) features both high resolution and high mass accuracy to measure the m/z of each ion to several decimal places.

“Lower limit of quantification” (LLOQ) refers to the lowest amount of an analyte that can be quantitatively determined with acceptable precision and accuracy.

“Below the limit of quantification” (BLQ) refers to an amount of an analyte that cannot be quantitatively determined with acceptable precision and accuracy.

“Liquid chromatography” (LC) refers to a method for the separation of mixtures. The mixture is dissolved in a fluid called the mobile phase (MP) which carries it through a structure called the stationary phase. .

“2D Chromatography” refers to a type of chromatography that separates a sample by passing through two different separation stages. The eluent from the first column is injected onto a second column that has a different separation mechanism. For example, a C18 column can be followed by a phenyl column or the two columns can be run at different temperatures.

“High performance liquid chromatography” (HPLC) refers to a method for separating, identifying, and quantifying each component in a mixture. It relies on pumps to pass a pressurized liquid solvent containing the sample mixture through a column filled with a solid adsorbent material. Each component in the sample interacts slightly differently with the adsorbent material, which causes different flow rates for the different components and leads to the separation of the components as they flow out of the column. HPLC differs from traditional LC by using higher operational pressures to pass the 1VIP (mobile phase) through a column.

A “C18 column” is a HPLC column that uses a C18 substance as the stationary phase.

An LC or HPLC system can contain various pumps and columns. For example, the LC system can contain a loading pump, a micro pump, and/or a nano pump. The LC system can also contain an analytical column and/or a trap column. The analytical column can be a nano liquid chromatography (nano-LC) column.

“Nano liquid chromatography” (Nano-LC) refers to LC that utilizes a nano-LC column with a decreased inner diameter to allow for a smaller sample amount, high efficiency, and increased sensitivity. A nano-LC column can have an internal diameter of 20-100 In some embodiments, the flow rate for the nano-LC is about 50˜about 500 nL/min (e.g., about 50˜about 100 nL/min, about 100˜about 150 nL/min, about 150˜about 200 nL/min, about 200˜about 250 nL/min, about 250˜about 300 nL/min, about 300˜about 350 nL/min, about 350˜about 400 nL/min, about 400˜about 450 nL/min, or about 450˜about 500 nL/min).

The terms “substantial” and “substantially” refer to a majority, i.e. >50% of a population, of a mixture or of a sample, preferably more than 50%, 55%, 60%, 65%, 70%, 75%, 80, 85%, 90%, 91%, 92%, 93%, 94%, 95%, 96%, 97%, 98%, or 99% of a population.

As used herein, the term “about” denotes an approximate range of plus or minus 10% from a specified value. For instance, the language “about 20 μg/Kg” encompasses a range of 18-22 μg/Kg. As used herein, “about” also includes the exact amount. Hence “about 20 μg/Kg” means “about 20 μg/Kg” and also “20 μg/Kg.”

As used herein, the term “agent” refers to a drug, label, toxin or the like.

In addition, designation of a range of values includes all individual values within or defining the range.

### Anti-CD40 Antibodies

CD40 is a member of the tumor necrosis factor (TNF) receptor superfamily. It is a single chain type I transmembrane protein with an apparent MW of 50 kDa. CD40 is expressed by some cancer cells, e.g., lymphoma cells and several types of solid tumor cells. CD40 also functions to activate the immune system by facilitating contact-dependent reciprocal interaction between antigen-presenting cells and T cells. See, e.g., van Kooten and Banchereau, J. Leukoc. Biol. 67:2-17 (2000); Elgueta et al., Immunol. Rev. 229:152-172 (2009).

Because of its role in immune function, antibodies have been raised against the CD40 antigen. Such antibodies can be classified into three groups: antagonistic antibodies, which inhibit CD40 activity; partially agonistic antibodies, which partially induce CD40 activity; and fully agonistic antibodies, which fully stimulate CD40 activity. Members of each of these groups have been tested in clinical trials; none have been approved to date. Anti-CD40 antibodies can contain fucose or can contain reduced or no fucosylation (i.e. non-fucosylated).

SEA-CD40 is a non-fucosylated humanized S2C6 antibody, also referred to herein as hS2C6. SEA-CD40 exhibits enhanced binding to FcγIII receptors, and surprsingly enhanced ability to activate the CD40 signaling pathway in immune cells. Because of its enhanced activation of the CD40 pathway, SEA-CD40 is a potent activator of the immune system and can be used to treat cancer or to treat infectious diseases, particularly chronic viral diseases, such as hepatitis C, human immunodeficiency virus, Epstein-Barr virus, cytomegalovirus, John Cunningham virus, and human papilloma virus. Other infectious diseases, include, e.g., tuberculosis. The enhanced activation of the immune system allows SEA-CD40 to be administered to patients at lower doses, using different schedules of administration.

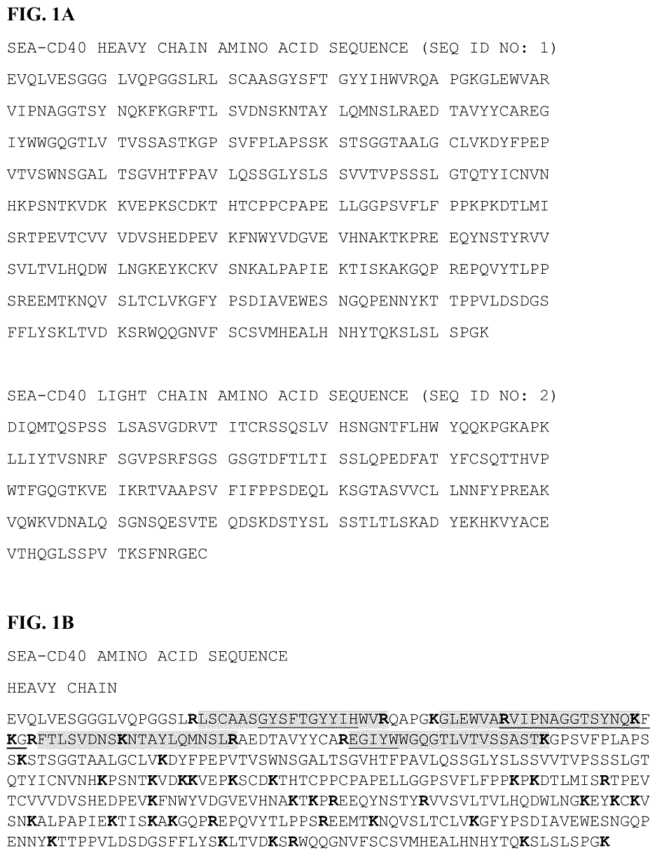

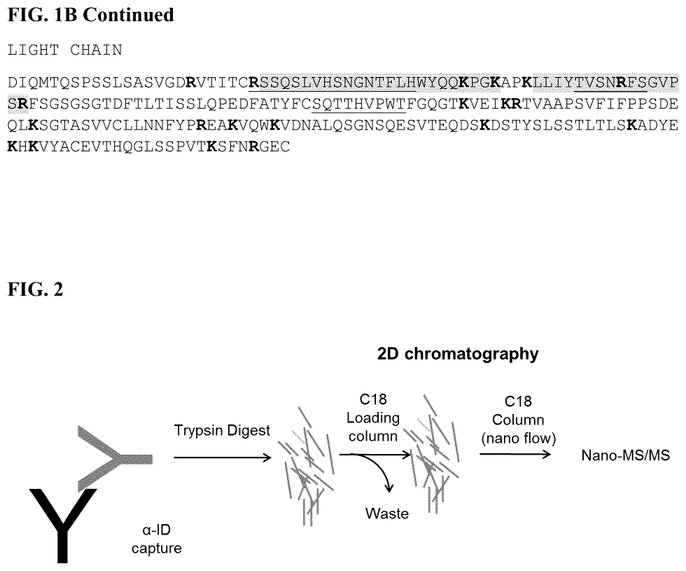

The amino acid sequences of the heavy and light chain for SEA-CD40 are disclosed as SEQ ID NOs: 1 and 2, respectively (see also FIG. 1A). The variable region (SEQ ID NO: 13) of the heavy chain is from amino acids 1-113 of SEQ ID NO:1. The variable region (SEQ ID NO: 14) of the light chain is from amino acids 1-113 of SEQ ID NO:2. The generation of the antibody backbone of SEA-CD40 is disclosed e.g., in WO 2006/128103, which is herein incorporated by reference.

The SEA-CD40 backbone contains reduced fucosylation of complex N-glycoside-linked sugar chains bound to the Fc region (or domain). Typically only a minor amount of fucose is incorporated into the complex N-glycoside-linked sugar chain(s) of the SEA-CD40 molecule. For example, in various embodiments, less than about 60%, less than about 50%, less than about 40%, less than about 30%, less than about 20%, less than about 15%, less than about 10%, less than about 5%, or less than about 3% of the antibody has N-glycoside-linked sugar chains that include a fucose residue. Preferably, the constant region of SEA-CD40 has an N-glycoside-linked sugar chain at residue N297 according to the EU index and less than 5% of the N-glycoside-linked sugar chains include a fucose residue, i.e., a fucose bound to the reducing terminal of the sugar chain via an α1,6 bond to N-acetylglucosamine (GlcNAc). Compositions and methods for preparing SEA-CD40 antibodies with reduced fucosylation are disclosed e.g., in WO 2016/69919, which is herein incorporated by reference. Those of skill will recognize that many methods are available to determine the amount of fucosylation on an antibody. Methods include, e.g., LC-MS via PLRP-S chromatography and electrospray ionization quadrupole TOF MS.

The anti-CD40 antibody for use in the methods of the present disclosure can include an anti-CD40 antibody with either the heavy chain variable region of SEQ ID NO: 13 or the light chain variable region of SEQ ID NO: 14. In some embodiments, the anti-CD40 antibody includes both the heavy chain variable region of SEQ ID NO: 13 and the light chain variable region of SEQ ID NO: 14. In some embodiments, the anti-CD40 antibody includes either the heavy chain of SEQ ID NO:1 or the light chain of SEQ ID NO:2. In some embodiments, the anti-CD40 antibody includes both the heavy chain of SEQ ID NO:1 and the light chain of SEQ ID NO:2.

In some embodiments, the heavy chain variable region (VH) comprises complementarity determining regions (CDRs) 1, 2, and 3, wherein the VH CDR1 region comprises an amino acid sequence that is at least 80%, 90% or 100% identical to SEQ ID NO: 15, the VH CDR2 region comprises an amino acid sequence that is at least 80%, 90% or 100% identical to SEQ ID NO: 16, and the VH CDR3 region comprises an amino acid sequence that is at least 80%, 90% or 100% identical to SEQ ID NO: 17; and a light chain variable region (VL) comprises CDRs 1, 2, and 3, wherein the VL CDR1 region comprises an amino acid sequence that is at least 80%, 90% or 100% identical to SEQ ID NO: 18, the VL CDR2 region comprises an amino acid sequence that is at least 80%, 90% or 100% identical to SEQ ID NO: 19, and the VL CDR3 region comprises an amino acid sequence that is at least 80%, 90% or 100% identical to SEQ ID NO: 20.

In some embodiments, the VH comprises an amino acid sequence that is at least 80%, 85%, 90%, 95% or 100% identical to SEQ ID NO: 13. In some embodiments, the VL comprises an amino acid sequence that is at least 80%, 85%, 90%, 95% or 100% identical to SEQ ID NO: 14.

In some embodiments, the heavy chain comprises an amino acid sequence that is at least 80%, 85%, 90%, 95% or 100% identical to SEQ ID NO: 1. In some embodiments, the light chain comprises an amino acid sequence that is at least 80%, 85%, 90%, 95% or 100% identical to SEQ ID NO: 2.

In some embodiments, the antibody or antigen-binding fragment thereof comprises a heavy chain variable region (VH) comprising VH CDR1, VH CDR2, and VH CDR3 that are identical to VH CDR1, VH CDR2, and VH CDR3 in SEQ ID NO: 1 or 13; and a light chain variable region (VL) comprising VL CDR1, VL CDR2, and VL CDR3 that are identical to VL CDR1, VL CDR2, and VL CDR3 in SEQ ID NO: 2 or 14.

The anti-CD40 antibody can also contain a human constant region. In some embodiments, the human constant region has an N-glycoside-linked sugar chain at residue N297 according to the EU index and less than 5% of the N-glycoside-linked sugar chains include a fucose residue. In some embodiments, the anti-CD40 antibody is non-fucosylated. In some embodiments, the anti-CD40 antibody is hS2C6 (i.e. SEA-CD40). In some embodiments, less than 5% of N-glycoside-linked sugar chains in the SEA-CD40 composition comprise a fucose residue.

### Analytic Peptides

The methods of the present disclosure include an anti-CD40 antibody that contains at least one analytic peptide. The at least one analytic peptide is preferably a unique signature peptide when searched against non-redundant or Swiss-Prot databases of predicted or known protein sequences. The search can use an algorithm for identifying unique peptide sequences, such as an NCBI Protein Blast algorithm.

The analytic peptide(s) can be contained within the heavy chain and/or the light chain of the anti-CD40 antibody. Particularly, the analytic peptide(s) is(are) contained within the heavy chain variable region and/or the light chain variable region. The analytic peptide(s) can also contain at least one amino acid residue of a CDR. In some embodiments, the analytic peptide contains a portion of or the entire amino acid sequence of a CDR.

An analytic peptide can also be a tryptic peptide. Accordingly, tryptic analytic peptides can be identified based on trypsin cleavage sites in an antibody amino acid sequence. FIG. 1B shows the Lysine (K) and Arginine (R) trypsin cleavage sites in the SEA-CD40 heavy and light chains in bold type font. CDR regions are underlined. Trypsin cleaves at the C-terminus of Arginine (R) and Lysine (K) residues. However, if a proline (P) residue occurs after a K or R residue, the site becomes a non-cleavage site.

In order to identify unique signature analytic peptides from anti-CD40 antibodies, an offline in silico digestion method can be used. Example 1 provides a method for use in some embodiments of the present disclosure for identifying unique signature analytic peptides from SEA-CD40 using an offline in silico tryptic digestion. With this method, tryptic peptides were identified within the heavy chain (SEQ ID NO: 1) and the light chain (SEQ ID NO: 2) of SEA-CD40 based on potential trypsin cleavage sites (Example 1.2). Highlighted amino acid sequences in FIG. 1B indicate identified unique tryptic peptides.

Even after unique analytic peptides have been identified, those peptides may not be suitable for use in identifying an anti-CD40 antibody in a sample. For example, analytic peptides containing a methionine (M) residue may not be suitable. Oxidation of a methionine (M) residue can lead to multiple forms (masses) for the same amino acid sequence that may confound quantification. In order to further identify a signature analytic peptide with high selectivity for use in the methods of the present disclosure, further optimization may be used. In some embodiments, such optimization can use LC-MS. By using LC-MS methodologies, those analytic peptides that exhibit poor MS fragmentation can be excluded as a signature peptide for use in the methods of the current disclosure.

Example 1.5 provides a method for use in some embodiments for the optimization of putative signature peptides. Such optimization can utilize spiking a high concentration of an anti-CD40 antibody into human plasma, which is then subjected to offline immunocapture with Protein G beads. Alternatively, a more targeted immunocapture strategy can involve using a biotinylated monoclonal antibody specific for the anti-CD40 antibody. The captured antibody can then be subject to trypsin digestion. Peptide selectivity can then be assessed by comparing blank matrix extraction to spiked plasma using LC-MS.

LC-MS/MS parameters are optimized to improve sensitivity and reproducibility. Such parameters can include ionization and fragmentation. In some embodiments, S-lens (ion source transmission) and/or collision energy (CE, peptide fragmentation) settings can be optimized. MS instrumentation for optimization can include the Thermo TSQ Vantage QQQ. Product ions can have different CE, thus software (e.g. Skyline) can be used to aid in precursor and product ion selection to help optimize CE. For example, Tables 2 and 3 identify the charge ion precursor selections for each signature peptide identified within SEQ ID NOs: 1 and 2. Peptides that are large in size or contain a non-cleavage site (KP), are identified as not being suitable for quantification due to poor fragmentation. Peptides with identifiable charge states can then be used in a method of the present disclosure for detecting and quantifying anti-CD40 antibodies.

In some embodiments, the analytic peptide for use in the methods of the disclosure can be contained within the heavy chain of SEQ ID NO: 1. Particularly, the analytic peptide is contained within the heavy chain variable region of SEQ ID NO: 13. Within SEQ ID NO: 13, the analytic peptide can have an amino acid sequence selected from: LSCAASGYSFTGYYIHWVR (SEQ ID NO: 3), GLEWVAR (SEQ ID NO: 4), VIPNAGGTSYNQK (SEQ ID NO: 5), and FTLSVDNS (SEQ ID NO: 6) (see Example 1.3 and Table 2).

In some embodiments, the analytic peptide can also be contained within the light chain of SEQ ID NO: 2. Particularly, the analytic peptide is contained within the light chain variable region of SEQ ID NO: 14. Within SEQ ID NO: 14, the analytic peptide can have an amino acid sequence selected from LLIYTVSNR (SEQ ID NO: 10) and FSGVPSR (SEQ ID NO: 11) (see Example 1.4 and Table 3). In particular, the analytic peptide can have the amino acid sequence LLIYTVSNR (SEQ ID NO: 10).

### Administration of Therapeutic Antibodies

Therapeutic antibodies can be administered to biological sources by any route appropriate to the condition to be treated. The antibody will typically be administered to a subject parenterally, e.g. infusion, subcutaneous, intramuscular, intravenous, intradermal, intrathecal and epidural. For administration of an antibody for the treatment of cancer, administration into the systemic circulation by intravenous or subcutaneous administration can be desired. For treatment of a cancer characterized by a solid tumor, administration can also be localized directly into the tumor, if so desired.

In some embodiments, the administration of the anti-CD40 antibody (e.g., SEA-CD40) is at a dose level between 0.1-2000 μg/kg (ug antibody per kilogram patient body weight). In one embodiment, the dose level is between 10-1000 μg/kg. In another embodiment, the dose level is between 50-800 μg/kg. In a further embodiment, the dose level is between 75-600 μg/kg. In another embodiment, the dose level is between 100-500 μg/kg. In further embodiments, the dose level is a range selected from the following: 100-300 μg/kg, 300-500 μg/kg, 500-700 μg/kg, 700-900 μg/kg, and 900-1100 μg/kg. In other embodiments, the dose level is a range selected from the following: 100-150 μg/kg, 150-200 μg/kg, 200-250 μg/kg, 250-300 μg/kg, 300-350 μg/kg, 350-400 μg/kg, 400-450 μg/kg, 450-500 μg/kg, 500-550 μg/kg, 550-600 μg/kg, 600-650 μg/kg, 650-700 μg/kg, 700-750 μg/kg, 750-800 μg/kg, 800-850 μg/kg, 850-900 μg/kg, 900-950 μg/kg, 950-1000 μg/kg, 1000-1050 μg/kg, and 1050-1100 μg/kg. In further embodiments, the dose level is selected from the following: about 60 μg/kg, about 100 μg/kg, about 150 μg/kg, about 200 μg/kg, about 250 μg/kg, about 300 μg/kg, about 350 μg/kg, about 400 μg/kg, about 450 μg/kg, about 500 μg/kg, about 550 μg/kg, about 600 μg/kg, about 650 μg/kg, about 700 μg/kg, about 750 μg/kg, about 800 μg/kg, about 850 μg/kg, about 900 μg/kg, about 950 μg/kg, about 1000-1050 μg/kg, about 1050 μg/kg, and 1110 μg/kg.

Preferably, an anti-CD40 antibody is administered intravenously. The antibody can also be administered subcutaneously at the site of a tumor. Methods of administering and dosing levels of SEA-CD40 antibodies are disclosed in WO 2016/69919, which is herein incorporated by reference. In some embodiments, SEA-CD40 can be administered to patients at levels between 0.1-2000 μg/kg (1.ig antibody per kilogram patient body weight). Other possible dosage ranges are 10-1000 μg/kg, 50-800 μg/kg, 75-600 μg/kg, 100-500 μg/kg. Other possible dosage ranges are the following: 100-300 μg/kg, 300-500 μg/kg, 500-700 μg/kg, 700-900 μg/kg, and 900-1100 μg/kg. Still more dose ranges are the following: 100-150 μg/kg, 150-200 μg/kg, 200-250 μg/kg, 250-300 μg/kg, 300-350 μg/kg, 350-400 μg/kg, 400-450 μg/kg, 450-500 μg/kg, 500-550 μg/kg, 550-600 μg/kg, 600-650 μg/kg, 650-700 μg/kg, 700-750 μg/kg, 750-800 μg/kg, 800-850 μg/kg, 850-900 μg/kg, 900-950 μg/kg, 950-1000 μg/kg, 1000-1050 μg/kg, and 1050-1100 μg/kg. Other possible dosage ranges are 0.3-200 μg/kg, 0.6-150 μg/kg, 1.0-100 μg/kg, 2-50 μg/kg, 5-25 μg/kg, 7.5-15 μg/kg, and 8-12 μg/kg. In some embodiments, SEA-CD40 is administered at 10 or 30 μg/kg.

In some embodiment, the dosing schedule is intravenous administration of SEA-CD40 (e.g., at 10 or 30 μg/kg) on days one and eight of a three week cycle. The total number of cycles can be determined by a physician. Another preferred dosing cycle is intravenous administration of SEA-CD40 (e.g., at 10 or 30 μg/kg) on day one of a three week cycle. The total number of cycles is determined by a physician. In some embodiments, intravenous administration of SEA-CD40 (e.g., at 10 or 30 μg/kg) is performed on days one and eight of a three week cycle for two cycles.

For subsequent cycles, SEA-CD40 is administered on day one of the three week cycle (e.g., at 10 or 30 μg/kg), with the total number of cycles determined by a physician. In some embodiments, SEA-CD40 is administered intravenously (IV) on a 21-day cycle with doses ranging from 0.6-60 μg/kg. In some embodiments, SEA-CD40 is administered IV in 21-day cycles (Day 1 of each cycle; Q3wk). The standard dosing regimen can be e.g., 0.6, 3, 10, 30, 45, or 60 μg/kg on Day 1. An intensified dosing regimen can also be used, wherein 30 μg/kg dosed on Day 1 and Day 8 of the first 2 cycles, with only one dose of SEA-CD40 administered on Day 1 in subsequent cycles.

### Hybrid Method for Quantifying Anti-CD40 Antibodies

### Obtaining a Sample Containing an Anti-CD40 Antibody

A sample for use in the methods of the present disclosure can include any volume of any medium that contains an amount of an anti-CD40 antibody. Preferably, the sample is at least or about 100 μL, at least or about 200 μL, at least or about 300 μL, at least or about 400 μL, at least or about 500 μL, at least or about 600 μL, at least or about 700 μL, at least or about 800 μL, at least or about 900 μL, or at least or about 1 ml. In some embodiments, the sample is less than 100, 200, 300, 400, 500, 600, 700, 800, 900, or 1000 μL.

The sample can be derived from any species (e.g. human, primate, mouse, rat, rabbit, etc.). In some embodiments, the sample is from a human. In some embodiments, the sample can be obtained from a patient that has been administered an anti-CD40 antibody. In some embodiments, the sample can be obtained from a human patient that has been administered an anti-CD40 antibody. The sample can be obtained from a patient at least once after administration of the anti-CD40 antibody. Two or more samples can be also obtained from a patient at different time points after administration of the anti-CD40 antibody. One of ordinary skill in the art can select a method appropriate for obtaining a sample containing an anti-CD40 antibody as described herein.

In some embodiments, the sample is plasma. When using a plasma sample, the sample can be treated with an anticoagulant to improve quantitative analysis. The anticoagulant can be ethylenediaminetetraacetic acid (EDTA) or dipotassium ethylenediaminetetraacetic acid (K2EDTA). Preferably, the plasma sample is treated with K2EDTA. The plasma sample can be obtained from any species. In particular, the plasma sample is from a human. In some embodiments, the plasma sample is from a patient that has been administered an anti-CD40 antibody. In some embodiments, the sample is plasma from a human patient that has been administered an anti-CD40 antibody. In some embodiments, the sample is collected from a human patient before or during the administration of the anti-CD40 antibody. In some embodiments, the sample is collected from a human patient at least or about 1, 2, 3, 4, 5, 6, 7, 8, 9, 10, 11, 12, 13, 14, 15, 16, 17, 18, 19, 20, 21, 22, 23, or 24 hours after the administration of the anti-CD40 antibody. In some embodiments, the sample is collected from a human patient at least or about 1, 2, 3, 4, 5, 6, 7, 8, 9, 10, 11, 12, 13, 14, 15, 16, 17, 18, 19, 20, or 21 days after the administration of the anti-CD40 antibody. One of ordinary skill in the art can select a method appropriate for obtaining a plasma sample containing an anti-CD40 antibody as described herein.

Extraction of the Anti-CD40 Antibody from the Sample

Once a sample has been obtained containing an anti-CD40 antibody, the method of the present disclosure can include extracting the anti-CD40 antibody from the sample. Extraction, for example, can include: affinity chromatography, size exclusion chromatography, ammonium sulfate precipitation, ion exchange chromatography, immobilized metal chelate chromatography, and immunoprecipitation. Particularly, extraction can include immunoaffinity chromatography. In some embodiments, extraction can include α-ID affinity capture using an α-ID antibody to the anti-CD40 antibody (see FIG. 2). The α-ID antibody can be labeled. Particularly, the α-ID antibody is labeled with biotin. A biotinylated α-ID anti-CD40 (anti-ID40) antibody can be prepared as described in Example 2.6.

Binding of an antibody to a material which contains a species to which the ligand or antibody binds can be used for extraction. Materials useful in extractions using these proteins include resins, e.g., beaded agarose, or magnetic beads, or similar support material to which protein A, protein G, protein L, or streptavidin is covalently immobilized. For example, extraction of an anti-CD40 antibody from a sample can involve adding protein G magnetic beads to the sample and thereafter removing the beads from the sample in order to capture the antibody, thereby extracting the antibody from the sample. Another example can involve capturing biotinylated antibodies with streptavidin magnetic beads. In some embodiments, the anti-CD40 antibody can be extracted by an anti-idiotypic antibody. The anti-idiotypic antibody can be labeled, e.g., with biotin. In some embodiments, the biotin-labeled anti-idiotypic antibody can specifically binds to the anti-CD40 antibody, and then streptavidin magnetic beads can be used capture the anti-idiotypic antibody along with the anti-CD40 antibody of interest. The structural requirements for binding of a given antibody to protein A, protein G or protein L are known in the art and one of ordinary skill in the art can select from among them to determine the appropriate surface protein for use with a given antibody.

In some embodiments, an anti-CD40 antibody is extracted from a sample as provided in Example 2.8. In further embodiments, the extraction methods are run in combination with control samples. The control samples can include standards, quality control (QC), dilution QC, control blank, and zero samples. In some embodiments, the QC samples are prepared according to Examples 2.2 and 2.5.

Release of the Analytic Peptide from the Anti-CD40 Antibody

After the anti-CD40 antibody has been extracted from the sample, the methods of the present disclosure can include releasing the analytic peptide from the anti-CD40 antibody. In some embodiments, the analytic peptide can be released with a proteolytic enzyme. Trypsin is a common proteolytic enzyme used to prepare a protein for analysis by mass spectrometry. In the present disclosure, some embodiments use a trypsin digest to release the analytic peptide from the anti-CD40 antibody (see FIG. 2).

Various trypsin digest conditions can be used in the methods of the present disclosure after immunoprecipitation of the anti-CD40 antibody (see Example 3). The conditions of the tryptic digestion can vary depending on factors, such as the amount of analyte, type of analyte, or the extraction procedure used to isolate an analyte from a sample. For example, the elution solution and buffer used after immunoprecipitation can affect tryptic digestion. Elution of the anti-CD40 antibody can include a solution of 10:90 acetonitrile:water (ACN):30 mM HCL or a solution of only 30 mM HC1. In some embodiments, an 30 mM HCl elution solution is used. The buffer used can contain 0.25 to 1.0 M Tris, pH 8.3. In some embodiments, the buffer contains 0.25 M Tris, pH 8.3. In some embodiments, the buffer contains 0.3 M Tris, pH 8.3. Various solutions for use in the present disclosure can be prepared as described in Example 2.7. In some embodiments, the solution comprises about 10 mM HCl to about 50 mM HCl (e.g., about 20 mM HCl to about 50 mM HCl, about 30 mM HCl to about 50 mM HCl, about 10 mM HCl to about 40 mM HCl, about 10 mM HCl to about 30 mM HCl, or about 20 mM HCl to about 40 mM HCl). In some embodiments, the acetonitrile:water ratio is about 50:50 to about 5:95, about 40:60 to about 5:95, about 30:70 to about 5:95, or about 20:80 to about 5:95.

Reduction and alkylation are typical steps following extraction to prepare a protein for MS analysis. Reducing disulfide bonds (e.g. with DTT or TCEP) and alkylating thiol groups (e.g. with iodoacetamide (IAA)) on a protein helps proteolytic enzymes to better gain access to cleavage sites on the protein. However, by avoiding reduction and alkylation, the salt load overall can be reduced and translate to better robustness of the assay. Accordingly, in some embodiments, the steps of extraction and release do not include steps of reduction and alkylation of the antibody.

Denaturation of the protein can also be conducted prior to tryptic digestion to expose the cleavage sites of the protein to the enzyme. Denaturation can be performed using urea or RapiGest (0.05% or 0.1%). However, denaturation can not necessarily help obtain a better signal via MS. Thus, in some embodiments, the steps of extraction and release do not include denaturation.

For the trypsin digestion, a tryptic digest solution containing trypsin can be used. A tryptic digestion solution containing trypsin and LysC can also be used. In some embodiments, the tryptic digestion solution contains about 10 to about 100 μg/mL, about 20 to about 90 μg/mL, about 30 to about 70 μg/mL, about 40 to about 60 μg/mL, about 50 to about 100 μg/mL of Trypsin and LysC. In some embodiments, the tryptic digestion solution contains about 50 μg/mL of Trypsin and LysC. The tryptic digestion solution can also contain other components. For example, the solution can also contain a resuspension buffer or water for reconstitution or dilution. A tryptic digestion solution for use in the methods as described herein can be prepared as provided in Example 2.7. In some embodiments, an analytic peptide is released according to the method provided in Example 2.8.

### Measuring the Amount of Analytic Peptide

### Liquid Chromatography

The methods of the present disclosure can include measuring the amount of analytic peptide after being released from the anti-CD40 antibody. In some embodiments, the present disclosure provides a system configured to perform a method described herein. In some of these embodiments, the system includes a means for separating an analytic peptide. In certain embodiments, the means includes liquid chromatography (LC). One of ordinary skill in the art can select a LC method appropriate for use in quantitation of analytic peptides in various samples as described herein.

The methods of the present disclosure can include LC. In some embodiments, the LC is HPLC. The LC system can contain various pumps and columns. For example, the LC system can contain a loading pump, a micro pump, and/or a nano pump. The LC or HPLC system can also contain an analytical column and/or a trap column. The analytical column can be a nano liquid chromatography (nano-LC) column. In some embodiments, the nano-LC column has an internal diameter of 20-100 μm. In some embodiments, the nano-LC column has an internal diameter of about or at least 75 μm. In some embodiments, the methods include a HPLC system with a nano-LC column and a trap column. In some embodiments, the LC system for use in the methods as described herein is provided in Example 2.9.

In some embodiments, the LC is 2D liquid chromatography. In 2D liquid chromatography, two different columns are connected in sequence and the effluent from the first column is transferred onto the second column. The analytic peptide in the second column can then be measured using a mass spectrometry method as described herein. In some embodiments, the first column is a loading column and the second column is a nano flow column. In some embodiments, the loading column and the nano flow column are both C18 columns (see FIG. 2).

### Mass Spectrometry

In some embodiments, the system includes a means for detecting a mass of an analytic peptide. In certain embodiments, the means includes a mass spectrometer. In preferred embodiments, tandem mass spectrometry (MS/MS) is used to quantify the amount of analytic peptide. MS/MS involves multiple steps of MS selection, with fragmentation occurring between the stages. In the first step of MS/MS, ions are formed in the ion source and separated by m/z ratio. In the second step of MS/MS, parent ions of a particular m/z ratio (precursor ions) are selected and fragment ions (product ions) are created, separated and detected. Parent ions can be fragmented to create fragment ions by collision-induced dissociation, ion-molecule reaction, photodissociation, or other process. In some embodiments, the fragment ions are created using collision-induced dissociation (CID). In further embodiments, the fragment ions are created using higher-energy collisional dissociation (HCD).

In MS/MS methods of the current disclosure, one or more fragment ions of a selected parent ion of the analytic peptide are monitored. A parent ion of the analytic peptide is selected as known in the art in the first MS step and that parent ion is subjected to fragmentation to generate one or more fragment ions each of which can be quantitated by measurement, for example, of the ion current associated with each fragment to generate ion current peaks as a function of mass (m/z). Integrated peak areas of a fragment can be measured for quantitation of the chemical species from which the parent ion and one or more fragment ions thereof derive. In measuring the analytic peptide herein, the one or more fragments are derived from the parent ion of the released analytic peptide.

A MS/MS method can be employed for quantitation of analytic peptides herein, and methods employing a triple quadrupole mass spectrometer are more typically employed for quantitative bioanlaysis. In one aspect, the MS/MS is performed on a TOF-MS, a Q-TOF, FTICR, Orbitrap, or high resolution ion-trap MS. Particularly, the MS is performed on an Orbitrap MS. In some embodiments, the MS/MS system for use in the methods as described herein is provided in Example 2.9.

Mass spectrometers used in the methods herein can be operated to monitor the entire mass spectrum of a sample, or more typically a selected portion thereof of interest. Particularly in MS/MS methods, the signal (e.g., ion current) from one or more fragment ions of a selected parent ion can be monitored. Selected reaction monitoring (SRM) operation can be used in which a single fragment ion generated from a selected parent ion is monitored. Alternatively, high resolution mass spectrometry (HRMS) can be used, which features both high resolution and high mass accuracy to measure the m/z of each ion to several decimal places. In some embodiments, the methods as described herein use HRMS (see Example 4).

### Liquid Chromatography-Mass Spectrometry

The methods of the present disclosure can use a combination of liquid chromatography with mass spectrometry (LC-MS). More specifically, the methods can use liquid chromatography tandem mass spectrometry (LC-MS/MS). In particular embodiments, the LC-MS/MS system uses nano-electrospray ionization with high resolution (LC-HRMS/MS). In some embodiments, the LC-MS/MS system for use in the methods as described herein is provided in Example 2.9.

### Electrospray Ionization (ESI)

Masses of relatively high molecular weight compounds, such as antibodies, can be detected at mass-to-charge ratios (m/z) that are easily determined by most mass spectrometers (typical m/z ranges of up to 2000, up to 3000, up to 8000). Electrospray ionization mass spectrometry (ESI-MS), in particular, is suited for charged, polar or basic compounds and for analyzing multiply charged compounds with excellent detection limits. In a standard ESI device, liquid containing an analyte of interest is pumped at flow rates of a few mL/min through a metal or glass capillary needle exposed to a high electric field. Exposure to the electric field disperses the liquid into an aerosol of fine charged droplets for detection by the mass spectrometer. When analyzed in the positive ion mode, protonated analyte molecules are observed in the mass spectrometer. When analyzed in the negative ion mode, deprotonated analyte molecules are observed.

In some embodiments, the method of the present disclosure utilizes nano electrospray ionization (nano-ESI). Nano-ESI only requires a few microliters (μL) of analyte solution and supports nL/min flow rates resulting in fine sprays and enhanced analytic sensitivity for molecular weight determination by MS/MS. Thus, nano-ESI is preferred for peptide and protein analysis because of the ability to analyze a small volume of sample. Nano-ESI can also utilize the positive or negative ion mode. In some embodiments, nano-ESI is utilized with LC-HRMS/MS in the positive ion mode.

Mass can be measured with a high resolution mass spectrometer. Each ion in the raw data that is related to a particular species in the deconvoluted mass spectrum has an intensity or abundance measure associated with it, and the abundance of all ions associated with a particular species thus constitutes an approximation of the abundance of that species in the sample that is analyzed. When species are quantitated, the abundance of a particular species in a sample is compared to the abundance of the species in a known sample, or calibration curve, which allows calculation of the quantity in the unknown sample.. Quantitation in this manner assumes that all species have approximately equivalent ionization efficiency. In some embodiments, the data from the nano-electrospray ionization LC/HRMS/MS can be handled as described in Example 2.11.

### Internal Standard (IS)

The precision of the methods of the present disclosure can be improved by including an internal standard (IS). The IS can be prepared using an isotopically labeled version of the analytic peptide, such that the IS can be detected independently in the LC-MS/MS system from the analytic peptide released from antibody. A known fixed amount of IS can be added to the sample. The concentration of IS can range from 0.100 to 50.0 ng/mL. Preferably, the concentration range is 0.5 to 50 ng/mL. The isotopically labeled IS can be added to each sample after the antibody has been extracted and immediately prior to releasing the analytic peptide from the antibody. More than one isotopic label can be incorporated into the IS. The isotopic label(s) can be stable or unstable. Preferably, the isotopic label(s) is(are) stable. The stable isotopic label(s) can be selected from C¹³ and N′ 5 . The quantitation of the analytic peptide can be then performed using the IS by LC-MS/MS techniques.

In some embodiments, the analytic peptide has the amino acid sequence LLIYTVSNR (SEQ ID NO: 10) and the internal standard has the amino acid sequence PGKAPKLLIYTVSNRFSGVPS (SEQ ID NO: 12). In particular embodiments, the IS can have the following structure: H2N-PGKAPKLLIYTV\hat{ }SNR\hat{ }FSGVPS-OH, wherein V\hat{ } and RA represent ¹³C and ¹⁵N labeled residues, respectively.

The quantitation analysis preferably includes calibration within the assay. A standard curve can be generated, for example, by preparing a series of samples with increasing concentrations of antibody. For example, calibration standard samples can have a concentration range of 0.5 ng/mL to 50.0 ng/mL. The increasing concentrations of calibration standard samples can also include one or more samples with concentrations of 0.500, 1.00, 2.00, 5.00, 10.0, 40.0, and 50.0 ng/ML. In some embodiments, calibration standard samples are prepared according to Examples 2.2 and 2.3 for use in the methods of the present disclosure. In some embodiments, the calibration standard curve can be generated as described in Example 2.3. In some embodiments, the calibration curve parameters can be established e.g., by linear regression, optionally with 1/x 2 weighting (Regression; response=slope x concentration+intercept). In some embodiments, the slope is about 0.23 to about 0.28 (e.g., about 0.24 to about 0.28, about 0.25 to about 0.28, about 0.24 to about 0.27, about 0.25 to about 0.27, about 0.26 to about 0.27, or about 0.265 to about 0.271). In some embodiments, the intercept is about −0.05 to about 0 (e.g., about −0.05 to about 0.1, about −0.04 to about 0, about −0.03 to about 0, about −0.02 to about 0, or about −0.01 to about 0).

The results for the same samples that are tested in different runs can be compared against each other to evaluate the accuracy. In some embodiments, the absolute value of relative error (RE) of the methods described herein is less than 1%, 2%, 3%, 4%, 5%, 6%, 7%, 8%, 9%, or 10%. In some embodiments, the coefficient of variation of the methods described herein is less than 1%, 2%, 3%, 4%, 5%, 6%, 7%, 8%, 9%, or 10%.

An IS can also be added to the standard curve samples, which are then processed by the LC-MS/MS method described above. The peak area for each standard is divided by the peak area obtained for the internal standard, and the resultant peak area ratios are plotted as a function of standard concentrations. The data points are fitted to a curve using, for example, linear regression analysis. In some embodiments, the internal standard solutions are prepared according to Example 2.4. In some embodiments, the peak area for an analytic peptide is divided by the peak area obtained for the internal standard, and the concentration calculations is based on this ratio.

The details of use of standard curves (calibration curves) as an external standard and a combination of the use of a standard curve with addition of internal standard for quantitation of analytes by MS, LC-MS and LC-MS/MS methods, are known in the art. One of ordinary skill in the art understands how to use such analytic methods in the determination of concentrations of analytes in a variety of samples, including biological samples as discussed herein.

Stable Isotope Labeling by/with Amino acids in Cell culture (SILAC)

The isotope-labeled anti-CD40 antibody (e.g., SEA-CD40) can be generated by various means. In some embodiments, the sequence encoding the antibody is cloned into appropriate vectors. The antibody can then be expressed in the presence of isotope labeled amino acid residues. For example, the cells can be fed with growth medium containing amino acids labeled with stable (non-radioactive) heavy isotopes, e.g., ¹³C and/or ¹⁵N. In some embodiments, the medium can contain an amino acid (e.g., aspartic acid, lysine and arginine) labeled with ¹³C. When the cells are growing in this medium, they incorporate the heavy amino acids into all of their proteins. Thereafter, all peptides containing the amino acids are heavier than their normal counterparts. Alternatively, uniform labeling with ¹³C or ¹⁵N can be used. The isotope-labeled proteins and unlabeled proteins can be combined and analyzed together by mass spectrometry. Pairs of peptides having the same sequences with or without isotope labels can be differentiated in a mass spectrometer owing to their mass difference. The ratio of peak intensities in the mass spectrum for such peptide pairs reflects the abundance ratio for the two proteins.

A pre-determined amount of isotope-labeled anti-CD40 antibody (e.g., SEA-CD40) can be added to the sample. The concentration of the isotope-labeled anti-CD40 antibody can range from 0.100 to 50.0 ng/mL. Preferably, the concentration range is 0.5 to 50 ng/mL. In some embodiments, the isotope-labeled anti-CD40 antibody can be added to each sample before extraction so that the isotope-labeled anti-CD40 antibody and the antibody in the samples can be subject to the same processes and will release the same analytic peptides. The quantitation of the analytic peptides can be then performed by LC-MS/MS techniques.

The isotope-labeled anti-CD40 antibody can also be added to the standard curve samples. In some embodiments, the peak area for each standard is divided by the peak area obtained for the isotope-labeled anti-CD40 antibody, and the resultant peak area ratios are plotted as a function of standard concentrations. The data points are fitted to a curve using, for example, linear regression analysis. In some embodiments, the concentration calculations is based on the ratio of peak areas.

### Determining the Amount of Anti-CD40 Antibody in a Sample

In the methods of the present disclosure, the amount of analytic peptide measured is a direct correlation to the amount of anti-CD40 antibody in the sample. Thus, the measured amount of analytic peptide determines the amount of anti-CD40 antibody in a sample. In some embodiments, the proportion of the amount of released analytic peptide in two or more samples at different time points determines the amount of anti-CD40 antibody in a patient over time, which can be used for different therapeutic methodologies (e.g. pharmacokinetics). In some embodiments, the proportion of the analytic peptide in two or more plasma samples over time determines the pharmacokinetics of the anti-CD40 antibody administered to a patient. In some embodiments, comparing the amount of the analytic peptide in two or more plasma samples over time, can be used to monitor the treatment of the patient administered with an anti-CD40 antibody.

### Pharmacokinetics

Monitoring circulating levels of a therapeutic antibody for pharmacokinetic (PK) determinations in a subject, including half-life, clearance, area under the curve (AUC), and volume of distribution, is necessary to establish safety/toxicity limits and appropriate dosing regimen (Welling, P. (1997) Pharmacokinetics Processes, Mathematics, and Applications, 2nd Ed., American Chemical Society, Washington, D.C.). Bioavailability is the extent to which the administered compound reaches general circulation from the administered dose form, usually expressed as a percentage of the administered dose. The half-life of a compound is the time required for 50% of the peak plasma concentration of the compound to be removed by excretion or biotransformation (metabolism). The therapeutic index expresses the selectivity of the compound between the desired therapeutic activity and the undesired toxic side effects. Mass spectrometry techniques for use in pharmacokinetics assays are known in the art. (See, e.g., Want et al., Spectroscopy 17:681-691 (2003); Okeley et al., Clin Cancer Res. 16: 888-897 (2010); Singh et al., DMD (2017); Alley et al., Bioconjugate Chem. 19:759-765 (2008)). The pharmacokinetic measurements from the methods as described herein elucidate the absorption, distribution, metabolism, and excretion of antibodies.

The methods described herein can be used in a variety of experiments that rely on the determination of the amount of anti-CD40 antibody in a sample. The methods herein can, for example, be used for determining the stability of administered therapeutic antibodies, and for studying the pharmacokinetics of a therapeutic antibody. The methods herein can be used to assess the use of therapeutic antibodies in clinical applications. In some embodiments, methods for determining the pharmacokinetics of an anti-CD40 antibody administered to a patient are provided. In some embodiments, methods for monitoring the treatment of a patent that has been administered an anti-CD40 antibody are provided.

### EXAMPLES

### Example 1: Identification of Unique Signature Peptides for SEA-CD40

### 1.1 Objective

An investigation was performed to determine whether SEA-CD40 contains a signature peptide for identification. A hybrid LC-MS approach was proposed featuring target protein immunocapture using magnetic beads and Hamilton Star robotics. SEA-CD40 was digested into peptides and analyzed using immunoaffinity chromatography coupled to nanoflow LC-MS/MS instrumentation.

### 1.2 Offline In Silico Digestion.

An offline in silico digestion of SEA-CD40 with trypsin enzyme was performed by e.g. Skyline. The in silico digestion identified tryptic peptides within the heavy chain (SEQ ID NO: 1) and the light chain (SEQ ID NO: 2) of SEA-CD40 based on potential trypsin cleavage sites. Trypsin cleaves at the C-terminus of arginine (R) and lysine (K) residues. However, if a proline (P) residue occurs after a K or R residue, the site becomes a non-cleavage site. FIG. 1B shows the lysine and arginine trypsin cleavage sites in the SEA-CD40 heavy and light chains in bold type font.

In identifying tryptic peptide sequences, those tryptic peptide sequences near CDR regions were preferable. CDR regions are indicated by underlines in FIG. 1B. Potential tryptic peptides were then searched against non-redundant or Swiss-Prot databases using an NCBI Protein BLAST algorithm to identify unique peptide sequence among predicted or known plasma protein background protein sequences. Amino acid sequences in grey color in FIG. 1B indicate unique tryptic peptides based on the BLAST search.

### 1.3 Heavy Chain Tryptic Products

Six unique peptides in the heavy chain (SEQ ID NO: 1) were identified from the BLAST search in Example 1.1 and are provided in Table 2. Underlined regions in the peptide amino acid sequences of Table 1 indicate CDR amino acid residues.

TABLE 2

| | Peptide | | | Use for |
|---|---|---|---|---|
| | Amino | | | selectivity |
| Pep- | Acid | SEQ | | in human |
| tide | Sequence | ID NO | Optimization | plasma |
| H1 | LSCAASGY | 3 | +3 charge state | Yes |
| | SFTGYYIH | | precursor | |
| | WVR | | (y3+, y4+, | |
| | | | y15++, y8+) | |
| H2 | GLEWVAR | 4 | +2 charge state | Yes |
| | | | precursor | |
| | | | (y3+, y4+, y5+) | |
| H3 | VIPNAGGT | 5 | +2 charge state | Yes |
| | SYNQK | | precursor | |
| | | | (y11++, y8+, | |
| | | | y9+) | |
| H4 | FTLSVDNS | 6 | +2 charge state | Yes |
| | | | precursor (y5+, | |
| | | | y6+, y7+) | |
| H5 | NTAYLQMN | 7 | Did not | No |
| | SLR | | optimize on | |
| | | | due to | |
| | | | methionine | |
| | | | residue | |
| | | | (bold font) | |
| H6 | EGIYWWGQ | 8 | Poor MS/MS | No |
| | GTLVTVSS | | fragmentation | |
| | ASTK | | | |

### 1.4 Light Chain Tryptic Products

Three unique peptides in the light chain (SEQ ID NO: 2) were identified in the BLAST search in Example 1.1 and are provided in Table 3. Underlined regions in the peptide amino acid sequences of Table 3 indicate CDR amino acid residues.

TABLE 3

| | Peptide | | | Use for |
|---|---|---|---|---|
| | Amino | | | selectivity |
| Pep- | Acid | SEQ | | in human |
| tide | Sequence | ID NO | Optimization | plasma |
| L1 | SSQSLVHS | 9 | Poor MS/MS | No |
| | NGNTFLHW | | fragmentation | |
| | YQQKPGK | | | |
| L2 | LLIYTVSN | 10 | +2 charge state | Yes |
| | R | | precursor (y5+, | |
| | | | y6+, y7+) | |
| L3 | FSGVPSR | 11 | +2 charge state | Yes |
| | | | precursor (y5+, | |
| | | | y6+) | |

### 1.5 LC-MS Optimization of Putative Signature Peptides

The heavy and light chain tryptic signature peptides identified in Tables 2 and 3 were subjected to LC-MS optimization in order to determine if the peptides were candidates for SEA-CD40 detection. Peptide H5 was not optimized due to the presence of the methionine (M) residue, which is indicated in bold type font in Table 2.

20 μL stock SEA-CD40 was digested with trypsin, diluted, and optimized with an ion trap mass spectrometer to elute nano LC-MS injections. The Thermo TSQ Vantage QQQ was used for the mass spectrometer. The optimized mass spectrometer settings include S-lens (source transmission) and collision energy (CE, peptide fragmentation). Skyline software was used to aid in precursor and product ion selection, which also helped to optimize CE. S-lens is a single value for the unfragmented precursor ion. Tables 2 and 3 indicate the charge ion precursor selection for each peptide.

Based on the optimization, H6, and L1 were found to have poor MS/MS fragmentation. When monitored by LC-MS, no significant peaks were identified. H6 may have had poor MS/MS fragmentation due to the large size of the peptide. L1 also may have had poor MS/MS due to the large size of the peptide. L1 also contains “KP” (bold type font), a non-cleavage site for trypsin, which could have also interfered with fragmentation. Peptides H1, H2, H3, H4, L2 and L3 were identified as candidates for SEA-CD40 detection.

### EXAMPLE 2: Quantitation of SEA-CD40 in Human Plasma by LC/HRMS/MS

### 2.1 Objective

A method for quantitative determination of SEA-CD40 via a tryptic signature peptide in human plasma samples was developed. The full amino acid sequence for SEA-CD40 is shown in FIG. 1A. Unique signature peptide sequences are highlighted within the full antibody sequence in FIG. 1B.

Human plasma samples containing SEA-CD40 were collected and mixed with dipotassium ethylenediaminetetraacetic acid (K2EDTA) anticoagulant in a tube. Blood plasma was then separated from the blood by centrifugation. The peptide L2 (LLIYTVSNR; SEQ ID NO: 10) (“L Peptide”), identified from Example 1.3, was selected as the tryptic signature peptide. The internal standard for SEA-CD40 (“L Peptide IS” or “IS”) was a stable isotope-labeled, extended amino acid sequence version of the signature peptide (H2N-PGKAPKLLIYTV\hat{ }SNR\hat{ }FSGVPS-OH (SEQ ID NO: 12), where V\hat{ } and R\hat{ } represent fully ¹³C and ¹⁵N labeled residues).

### 2.2 Sample Preparation Procedure

Calibration standard (STD) samples were prepared fresh in human plasma on the day of analysis. For each regressed run, two STD samples at each concentration level were prepared and extracted. The STD concentrations were 0.500, 1.00, 2.00, 5.00, 10.0, 40.0, and 50.0 ng/mL for SEA-CD40.

QC samples were prepared in human plasma and stored at −20° C. and −70° C. For LLOQ QC, QC1 (Low), QC3 (Medium), and QC4 (High), the concentrations were 0.500, 1.50, 25.0, and 38.0 ng/mL, respectively. Geometric mean QC samples (QC2 or GMQC; 6.00 ng/mL) were not required for validation but will be qualified for use if needed for sample analysis. Dilution QC (DilQC/QC5; 250 ng/mL) samples were prepared to exceed the upper limit of quantitation (ULOQ) and analyzed using 10-fold dilution.

A 96-well immunoprecipitation extraction procedure with an anti-idiotypic antibody for SEA-CD40, with subsequent addition of IS, was used to isolate SEA-CD40 and the IS from K₂EDTA human plasma samples (200-μL sample aliquots).

### 2.3 Preparation of Calibration Standard Curve

Milli-Q water (18 megaohm-cm) or equivalent was used. Different volumes of calibration standard solutions may be prepared as applicable. All solutions were mixed well. Calibration standard stock and working solutions were stored at −70° C. and brought to room temperature before use.

### Preparation of Calibration Standard Stock and Working Solutions

Standard Stock Solution (42.1 mg/mL SEA-CD40 in 10 mM Histidine, 8.0% Trehalose, 0.2 mg/mL PS20, pH 5.5) was received in solution at 42.1 mg/mL in 10 mM histidine, 8.0% trehalose, 0.2 mg/mL PS20, pH 5.5.

Intermediate Standard Working Solution (0.5 mg/mL SEA-CD40 in 5% BSA in 10 mM PBS) was created by combining 20 μL of SEA-CD40 Stock Solution and 1.664 mL of 5% BSA in 10 mM PBS in a 2-mL Protein LoBind tube to yield a 0.5-mg/mL intermediate solution. The solution was then vortexed to mix and stored in single-use aliquots, 50 μL per tube.

Preparation of Standard Curve

The Standard Working Solution (SWS) and all calibration standards directly in 2-mL Protein LoBind tubes were prepared as described below and prepared fresh on each day of analysis and discarded after use. The system suitability sample was prepared by combining 100μL each of STD1 and STD2 in the sample well.

TABLE 4

| | Concentration | | |
|---|---|---|---|
| Standard | of SEA-CD40 | Volume and | Volume of |
| ID | (ng/mL) | Solution Used | Matrix (μL) |
| SWS | 2500 | 10 μL of SWS | 1990 |
| | | Intermediate | |
| STD7 | 50.0 | 40 μL of SWS | 1960 |
| STD6 | 40.0 | 16 μL of SWS | 984 |
| STD5 | 10.0 | 300 μL of STD7 | 1200 |
| STD4 | 5.00 | 100 μL of STD7 | 900 |
| STD3 | 2.00 | 40 μL of STD7 | 960 |
| STD2 | 1.00 | 20 μL of STD7 | 980 |
| STD1 | 0.500 | 50 μL of STD 5 | 950 |

### 2.4 Preparation of Internal Standard Solutions

Internal Standard solutions were prepared fresh on each day of analysis and discarded after use. Different volumes may be prepared as applicable.

### Preparation of IS Stock and Working Solutions

IS Stock Solution (1 mg/mL L Peptide IS in 30:70 Acetonitrile/Water) was prepared by weighing approximately 0.5 mg of L peptide IS on a microbalance and then transferring to a polypropylene tube. An appropriate volume of 30:70 acetonitrile/water was added to yield a 1-mg/mL solution, and then sonicated for 5 minutes and vortexed for 1 minute. The solution was stored at 4° C. and brought to room temperature before use.

IS Working Solution Intermediate I (5 μg/mL L Peptide IS in 30:70 Acetonitrile/Water) was created by combining 10 μL of the L Peptide IS Stock Solution and 1.990 mL of 30:70 acetonitrile/water in a 2-mL Protein LoBind tube to yield a 5-μg/mL intermediate solution. The solution was then vortexed to mix and stored in single-use aliquots, 50 μL per tube, at −70° C. and brought to room temperature before use.

IS Working Solution Intermediate II (500 ng/mL L Peptide IS in 30:70 Acetonitrile/Water) was created by combining 10 μL of the IS Working Solution Intermediate I and 90 μL of 30:70 acetonitrile/water in a 1.5-mL Protein LoBind tube to yield a 500-ng/mL intermediate solution. The solution was prepared fresh as needed and discarded immediately after use.

IS Working Solution (1 ng/mL L Peptide IS in 30:70 Acetonitrile/Water) was created by combining 10 μL of the IS Working Solution Intermediate II and 4990 μL of 30:70 acetonitrile/water in a polypropylene tube to yield a 1-ng/mL intermediate solution. The solution was prepared fresh as needed and discarded immediately after use.

### 2.5 Preparation of Quality Control Solutions

Quality control (QC) solutions were stored at −70° C. and brought to room temperature before use. Different volumes of QC solutions may be prepared as applicable.

### Preparation of QC Stock and Working Solutions

QC Stock Solution (42.1 mg/mL SEA-CD40 in 10 mM Histidine, 8.0% Trehalose, 0.2 mg/mL PS20, pH 5.5) was received in solution at 42.1 mg/mL in 10 mM histidine, 8.0% trehalose, 0.2 mg/mL PS20, pH 5.5.

Intermediate QC Working Solution (0.5 mg/mL SEA-CD40 in 5% BSA in 10 mM PBS) was prepared in the same fashion as the SEA-CD40 Intermediate Standard Working Solution.

### Preparation of QC Samples

QC Working Solution (QCWS) was prepared directly in polypropylene tubes. All QC samples were prepared from the QCWS, DilQC, or QC3 directly in polypropylene tubes. The dilution QC (250 ng/mL SEA-CD40) was prepared as a 10-fold dilution by adding 20 μL of DilQC to 180 μL of control plasma. A 200-μL aliquot was analyzed.

### 2.6 Preparation of Biotinylated Antibody

250 μL of 9.98 mg/mL anti-ID40 mAb antibody solution was mixed with 1.372 mL of 10 mM PBS buffer (pH 7.4), and 41.6 μL of 10 mM Sulfo-NHS-LC-Biotin. The mixture was incubated at 700 rpm at room temperature for 2 hours, and then was desalted by Zeba Desalt Spin column. The biotin-labeled anti-ID40 mAb antibody was then stored for the following experiments.

### 2. 7 Preparation of Other Solutions

Milli-Q water (18 megaohm-cm) or equivalent was used. Different volumes may be prepared as applicable. All solutions were mixed thoroughly and stored at room temperature unless otherwise noted.

30:70 Acetonitrile/Water (Diluent) was created by combining 150 mL of acetonitrile (NOWPak) and 350 mL of water in a 500-mL bottle.

Biotinylated Antibody Working Solution (60 μg/mL) was created by adding 1.920 mL of 10 mM phosphate buffered saline, pH 7.4, to an 80-μL frozen aliquot of 1.5-mg/mL anti-SEA-CD40 biotinylated antibody solution. Prepared one vial per 96-well plate immediately before use and discarded after use.

0.3 M Tris pH 8.3 Neutralizing Buffer was created by mixing 9 mL of 1 M Tris pH 8.3 buffer with 21 mL of water. Prepared fresh on day of analysis and discarded after use.

Trypsin/Lys C Solution (50 μg/mL) was created by reconstituting 100 1.tg of Trypsin/Lys C with 500 μL of resuspension solution. Added 1.5 mL of water to dilute to 501.tg/mL to produce four vials. Prepared two vials per 96-well plate immediately before use and discarded after use.

CHAPS Buffer was created by adding 15.48 g ammonium acetate, 28.4 g sodium chloride, and 2.00g of CHAPS to a 2-L volumetric flask partially filled with water and dissolving completely. The solution was then brought to volume with water, mixed by shaking, and transferred to a 2-L glass bottle with screw cap. The solution was stored at 4° C.

10 mM PBS Buffer was created by combining 2 L of water and 10 tablets of PBS in a 2-L bottle. The solution was sonicated to dissolve and mixed well. The solution was stored at 4° C.

5% PBS buffer in 10 mM PBS was created by adding 12.9g of B SA to a 250 mL volumetric flask containing 10 mM PBS buffer and brought to volume with PBS buffer. The solution was dissolved by shaking and transferred to polypropylene tubes. The solution was stored at −20° C.

0.5% BSA in 10 mM PBS, pH 7.4 was created by diluting 15 mL of 5% BSA in 10 mM PBS, pH 7.4, with 135 mL of 10 mM PBS, pH 7.4. Preparation is for one 96-well plate; double amounts for two plates. The solution was discarded after use.

1 M Hydrochloric Acid Solution was created by adding 8.212 mL of hydrochloric acid to a 100-mL volumetric flask partially filled with water. The solution was brought to volume with water, mixed well and transferred to a glass bottle. The solution was stored at 4° C.

30 mM Hydrochloric Acid Solution was created by adding 15 mL of 1 M hydrochloric acid solution to a 500-mL volumetric flask partially filled with water. The solution was brought to volume with water, mixed well and transferred to a glass bottle.

20% Formic Acid in Water was created by adding 20 mL of formic acid to a 100-mL volumetric flask containing water and brought to volume with water.

50:50 Isopropyl alcohol/Water (Autosampler Wash) was created by combining 1000 mL of isopropyl alcohol and 1000 mL of water in a 2-L glass bottle. The solution was mixed by shaking and sonicated to degas.

600:300:100:10 Isopropyl alcohol/Acetonitrile/Water/Formic Acid (Left Pump Mobile Phase C) was created by combining 1200 mL of isopropyl alcohol, 600 mL of acetonitrile, 200 mL of water, and 20 mL of formic acid in a 2-L glass bottle. The solution was mixed and sonicated.

10:1000 Formic Acid/Water (Right Pump Mobile Phase B) was created by combining 20 mL of formic acid and 2000 mL of water in a 2-L glass bottle. The solution was mixed by shaking.

2% Acetonitrile, 0.1% Formic Acid in Water (Nano Pump Mobile Phase A) was created by combining 1960 mL of water and 40 mL of acetonitrile in a 2-L glass bottle. 2 mL of formic acid was added and mixed well. The solution was sonicated with vacuum.

90% Acetonitrile, 0.1% Formic Acid in Water (Nano Pump Mobile Phase B) was created by combining 1800 mL of acetonitrile and 200 mL of water in a 2-L glass bottle. 2 mL of formic acid was added and mixed well. The solution was sonicated with vacuum.

### 2.8 Sample Extraction Procedure

Samples were extracted using an immunoprecipitation extraction procedure. Analytical runs are prepared with study samples, STD1 through STD7 (n=2), QC samples 1 through 4 (n≥3), dilution QC samples (n≥3, if applicable), control blank samples, and zero samples. One carryover zero sample and ≥1 carryover mitigation zero samples should be placed after each calibration curve in the run.

Extraction recovery of SEA-CD40 from human plasma can be determined by comparing the peak area ratios (PAR) for SEA-CD40 in samples spiked after extraction (post-extract) with the PAR of samples spiked before extraction (pre-extract). SEA-CD40 and IS solutions were added to the post-extract samples following the immunoprecipitation step and prior to the addition of trypsin/Lys C solution.
1. Prepared the calibration standards and quality control samples as outlined at room temperature.
2. Aliquoted 200 μL each of sample, standard, QC, control blank, or zero sample directly into the wells of a 2-mL 96-well Protein LoBind plate (Plate A).
3. Prepared the system suitability sample by combining 100 μL of STD1 and 100 μL of STD2 into the applicable well of Plate A.
4. Added 1300 μL of 0.5% BSA in 10 mM PBS, pH 7.4 to each well of Plate A. Added to the sides of the well to avoid splashing.
5. Added 20 μL of biotinylated antibody working solution to each well.
6. Sealed with cap mat and centrifuged for 1 minute at 1000× g.
7. Shook in chilled incubator at approximately 800 rpm for 1 hour.
8. During the 1-hour incubation, gently suspended Streptavidin Ti beads using a shaker set to 600 rpm for approximately 20 minutes without vortexing.
9. Centrifuged Plate A for 1 minute at 1,000× g.
10. Added 20 μL of suspended Streptavidin Ti beads to Plate A.
11. Incubated Plate A at room temperature for 30 minutes at 800 rpm to allow the Streptavidin Ti beads to bind up biotinylated antibody.

### Steps 12 to 20 Were Performed on the Hamilton STAR

12. Transferred sample and beads from Plate A to a 0.5 mL Protein Lobind collection plate (Plate B) in 6 x 280-μL transfers. During each transfer Plate B was allowed to rest on a magnetic stand and supernatant was aspirated to waste.
13. Added 280 μL of CHAPS buffer to Plate A to collect residual beads.
14. Transferred the 280 μL CHAPS buffer containing residual beads from Plate A to Plate B.
15. Allowed the Streptavidin Ti beads to migrate to the sides of the wells and aspirated CHAPS buffer supernatant to waste.
16. Washed the beads 2 times with 280 μL of CHAPS buffer and aspirated CHAPS buffer to waste.
17. Washed the beads with 280 μL of 10 mM PBS buffer and aspirated PBS buffer to waste.
18. Added 0.30 M Tris, pH 8.3 neutralizing solution and 30 mM hydrochloric acid solution to the deck.
19. Added 30 μL of 0.30 M Tris, pH 8.3 to a 1-mL Protein Lobind elution plate (Plate C).
20. Performed this step 2 times. Added 70 μL of 30 mM hydrochloric acid solution to Plate B, mixed for 3 minutes, and transferred the supernatant from Plate B to Plate C.

### Steps 21 to 28 Were Performed by the Analyst

21. Removed Plate C from the STAR and returned to the bench.
22. Added 30 μL of IS (internal standard) working solution to each sample well using a repeater pipette. Added 30 μL of 30:70 acetonitrile/water to control blanks.
23. Prepared the 50-μg/mL Trypsin/Lys C solution.
24. Added 40 μL of 50-μg/mL Trypsin/Lys C solution to each well and mixed for 1 minute at 800 rpm on the Mixmate.
25. Sealed with plastic capmat and centrifuged for 1 minute at 1,000× g.
26. Shook in a 37° C. oven overnight.
27. Added 20 μL of 20% formic acid into each well and sealed with metal foil.
28. Centrifuged for 1 minute at 1,000× g.
29. Shook at 1200 rpm at room temperature for 3 minutes.

### 2.9 LC-MS/MS Instrumentation

The 200 μL sample aliquots were analyzed by liquid chromatography/high resolution mass spectrometry (LC-HRMS) in the positive ion mode. The liquid chromatography/mass spectrometry (LC-MS) system consisted of a high-performance liquid chromatography (HPLC) Dionex UltiMate system (WPS-3000TPL Autosampler with a 250-μL sample loop, DGP-3600RS Dual-Gradient Rapid Separation Pump with left and right pumps, NCS-3500RS Binary Rapid Separation Nano/Capillary Pumps; and TCC-3000RS column oven) (Thermo Scientific), a trap column at 60° C. (μ-Precolumn Cartridge fitted with an Acclaim PepMap100 C₁₈, 5 μm, 100 ∪ 300 μm i.d.×5 mm) (Thermo Scientific), a nano LC column at 60° C. (EASY-Spray PepMap C₁₈, 75 μm×15 cm) (Thermo Scientific), and a Q Exactive Plus Hybrid Quadrupole-Orbitrap Mass Spectrometer (Thermo Scientific).

### HPLC Conditions

### Loading Pump (Left Pump) Mobile Phases and Program:

Mobile Phase C: 600:300:100:10 Isopropyl alcohol/Acetonitrile/Water/Formic Acid

TABLE 5

| Retention | Flow rate | | | |
|---|---|---|---|---|
| Time (min) | (mL/min) | % A | % B | % C |
| 0.00 | 0.05 | 0 | 0 | 100 |
| 7.00 | 0.05 | 0 | 0 | 100 |
| 7.10 | 0.25 | 0 | 0 | 100 |
| 11.10 | 0.25 | 0 | 0 | 100 |

### MicroPump 1 (Right Pump) Mobile Phases and Program:

Mobile Phase B: 10:1000 Formic Acid/Water

TABLE 6

| Retention | Flow rate | | | |
|---|---|---|---|---|
| Time (min) | (mL/min) | % A | % B | % C |
| 0.00 | 0.300 | 0 | 100 | 0 |
| 4.0 | 0.300 | 0 | 100 | 0 |
| 4.1 | 0.050 | 0 | 100 | 0 |
| 8.1 | 0.050 | 0 | 100 | 0 |
| 8.2 | 0.300 | 0 | 100 | 0 |
| 11.10 | 0.300 | 0 | 100 | 0 |

### Nano Pump Mobile Phases and Program:

Mobile Phase A: 2% Acetonitrile, 0.1% Formic Acid in Water
Mobile Phase B: 90% Acetonitrile, 0.1% Formic Acid in Water.

TABLE 7

| Retention | Flow rate | |
|---|---|---|
| Time (min) | (mL/min) | % B |
| 0.00 | 0.600 | 10 |
| 3.5 | 0.600 | 10 |
| 7.5 | 0.600 | 40 |
| 7.6 | 0.600 | 90 |
| 9.5 | 0.600 | 90 |
| 9.6 | 0.600 | 10 |
| 11.10 | 0.600 | 10 |

### Mass Spectrometry

### Non-Tunable Parameters:

Mass Spectrometer: Thermo Q Exactive Plus Series Mass Spectrometer (LCquan v 2.9)
Ionization: Thermo EASY-Spray
Ionization Mode: Positive Ion
Scan Type: High mass resolution, accurate mass spectrometry; parallel reaction monitoring (MS/MS)
Acquisition Time: 6 minutes
Minimum Cycle Time: Approximately 12 minutes
Orbitrap Resolution: 17,500
Quadrupole Isolation Width: 1.0 m/z units
NCE: 17 for both analyte and IS
AGC Target: 5×10⁵
Maximum Injection Time: 50 ms
Spectrum Data: Profile
Compounds: Charge State (z=+2)
SEA-CD40 539.8
L peptide IS 547.8

### Tunable Parameters:

Spray Voltage: 2.3 kV
Capillary Temperature: 275° C.
S-Lens RF Level: 90

### Expected Retention Time:

SEA-CD40: 4.2±0.8 minutes
L peptide IS: 4.2±0.8 minutes

### 2.10 Mass Spectrometric and Chromatographic Characterization

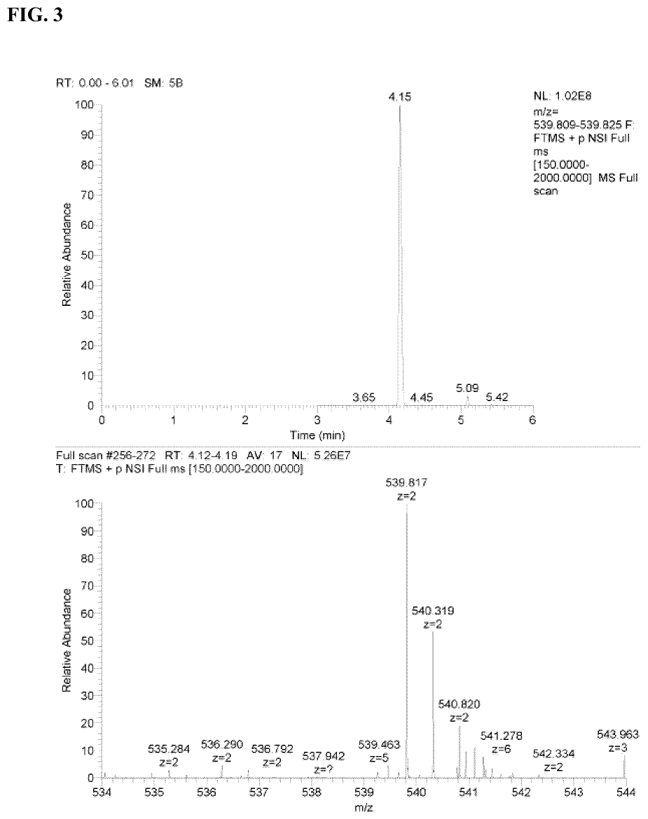

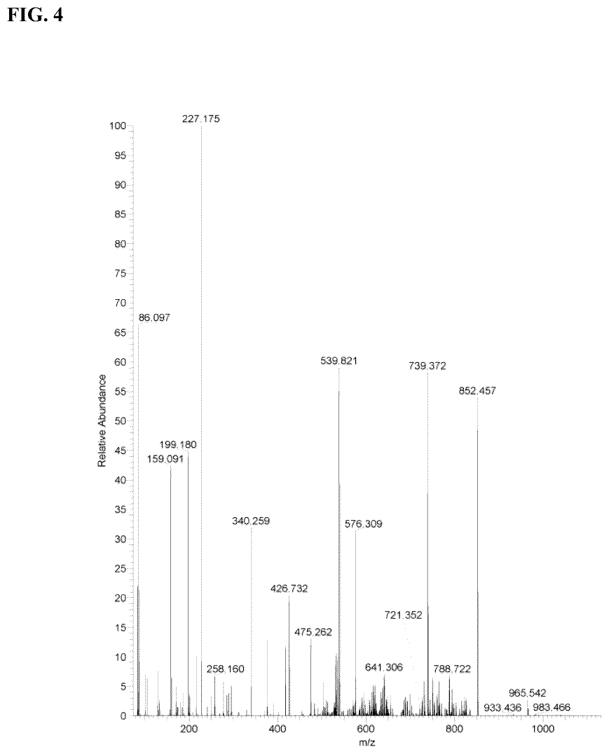

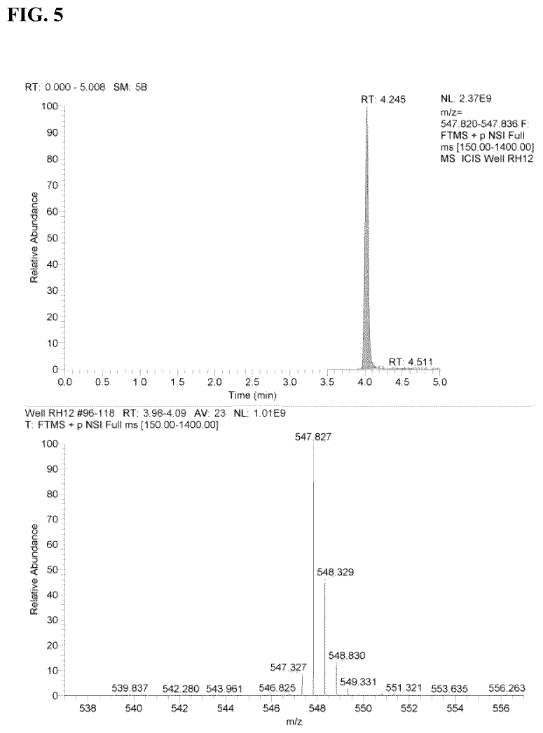

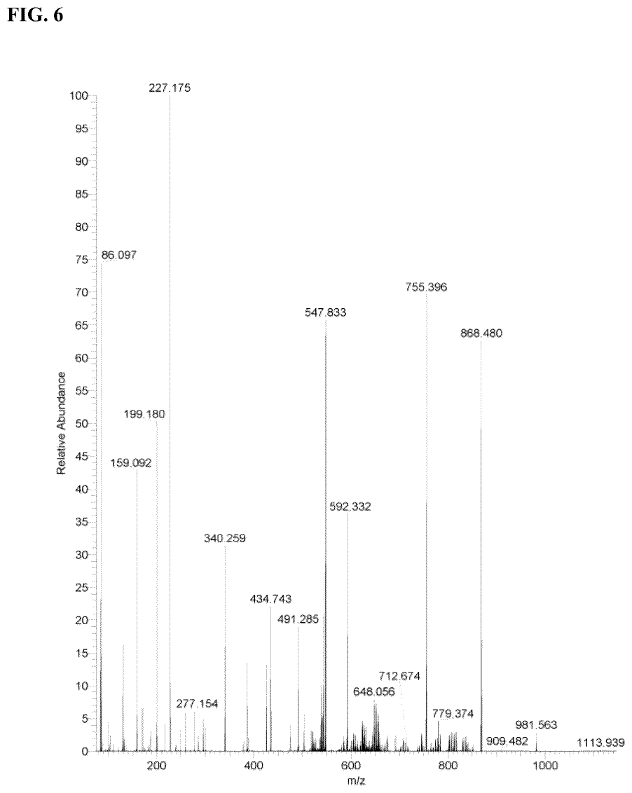

Representative full-scan MS1 and full-scan higher-energy collisional dissociation (HCD) product ion mass spectra for SEA-CD40 and the IS were acquired by injection and are shown in FIGS. 3-6. Based on the mass spectral characterization, the following product ions were monitored for data acquisition:

| | |
|---|---|
| SEA-CD40 | m/z 739.373, 852.457 (z = +1) |
| Internal | m/z 755.396 (z = +1) |
| Standard | |

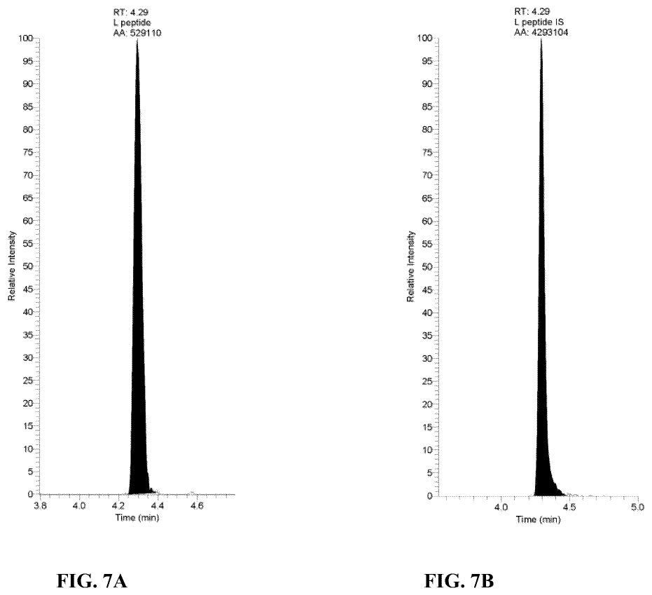

Representative chromatograms from a control blank (matrix only), zero sample (IS only), STD1 (LLOQ) are shown in FIGS. 7A and 7B, respectively.

### 2.11 Data Handling

The data were collected using nano-electrospray ionization LC/HRMS/MS in the positive ion mode. Peak areas were integrated on a Windows 7 platform by the Thermo program LCquan, v 2.9. Following peak area integration, the results tables from LCquan were saved as text files and uploaded to the Q2 Solutions file server where a weighted (1/x2) linear regression for SEA-CD40 was performed using the laboratory information management system (LIMS), Watson v 7.4.2 (Thermo Fisher Scientific Inc.). All concentration calculations were based on the peak area ratios (PAR) of SEA-CD40 to the IS. Concentrations of the analyte in QC samples were determined by back-calculation from the calibration curve.

Concentration data and statistics were downloaded from Watson. Excel (Microsoft) was used to perform calculations for results of various experiments when Watson could not be used. Final concentrations were reported to three significant figures and accuracy and precision values were reported to one decimal place.

### 2.12 Results

### Calibration Standard Curve

The calibration curve parameters for SEA-CD40 in human plasma is shown in the table below. The calibration range of 0.500 to 50.0ng/mL was established with linear regression and 1/x² weighting (Regression; response=slope×concentration+intercept).

TABLE 8

| Run | | | | LLOQ | ULOQ |
|---|---|---|---|---|---|
| No. | Slope | Intercept | r-Squared | (ng/mL) | (ng/mL) |
| 1 | 0.270627 | −0.017862 | 0.9955 | 0.500 | 50.0 |
| 2 | 0.269275 | −0.002991 | 0.9959 | 0.500 | 50.0 |
| 3 | 0.269083 | −0.009393 | 0.9957 | 0.500 | 50.0 |
| 4 | 0.261367 | −0.010909 | 0.9930 | 0.500 | 50.0 |
| 5 | 0.246285 | −0.001053 | 0.9985 | 0.500 | 50.0 |
| 7 | 0.236687 | −0.042500 | 0.9919 | 0.500 | 50.0 |
| 8 | 0.236257 | −0.005399 | 0.9951 | 0.500 | 50.0 |

The calibration standard concentrations for SEA-CD40 in human plasma is shown in the table below. Back-calculated concentrations within ±20.0% (±25.0% at LLOQ) of nominal for all regressed and accepted runs.

Accuracy and precision are defined as follows:
Accuracy is expressed as relative error (RE).

RE=[(Mean−Nominal)/Nominal]×100

Precision is expressed as coefficient of variation (CV).

CV=(Standard Deviation/Mean)×100

TABLE 9

| | SEA-CD40 Concentration | | | | | | |
|---|---|---|---|---|---|---|---|
| Run | STD1 | STD2 | STD3 | STD4 | STD5 | STD6 | STD7 |
| No. | 0.500 ng/mL | 1.00 ng/mL | 2.00 ng/mL | 5.00 ng/mL | 10.0 ng/mL | 40.0 ng/mL | 50.0 ng/mL |
| 1 | 0.521 | 0.913 | 1.92 | 4.53 | 10.6 | 42.9 | 48.9 |
| | 0.517 | 0.988 | 1.90 | 4.92 | 10.1 | 42.0 | 52.7 |
| 2 | 0.510 | 0.963 | 1.99 | 4.99 | 10.2 | 42.8 | 54.3 |
| | 0.520 | 0.944 | 1.97 | 4.41 | 9.95 | 40.8 | 48.8 |
| 3 | 0.497 | 1.04 | 2.07 | 5.59 | 10.3 | 41.1 | 49.7 |
| | 0.498 | 0.983 | 1.86 | 4.68 | 10.0 | 36.3 | 50.4 |
| 4 | 0.511 | 0.937 | 1.98 | 4.54 | 10.3 | 45.5 | 47.8 |
| | 0.535 | 0.923 | 1.85 | 4.99 | 10.9 | 40.7 | 49.3 |
| 5 | 0.474 | 1.02 | 1.97 | 5.09 | 9.62 | 41.5 | 51.5 |
| | 0.523 | 0.989 | 2.05 | 4.89 | 9.73 | 38.8 | 50.5 |
| 7 | 0.500 | 0.982 | 1.93 | 4.31 | 9.29 | 40.1 | 48.0 |
| | 0.477 | 1.14 | 2.11 | 4.95 | 9.90 | 44.7 | 53.1 |
| 8 | 0.476 | 1.00 | 2.08 | 5.14 | 9.85 | 42.5 | 45.8 |
| | 0.511 | 1.01 | 2.01 | 5.22 | 10.9 | 35.4 | 47.9 |
| Mean | 0.505 | 0.988 | 1.98 | 4.88 | 10.1 | 41.1 | 49.9 |
| SD | 0.0190 | 0.0572 | 0.0808 | 0.349 | 0.462 | 2.82 | 2.35 |
| CV (%) | 3.8 | 5.8 | 4.1 | 7.2 | 4.6 | 6.9 | 4.7 |
| RE (%) | 1.0 | −1.2 | −1.0 | −2.4 | 1.0 | 2.8 | −0.2 |
| n | 14 | 14 | 14 | 14 | 14 | 14 | 14 |

### Accuracy and Precision of Quality Control Samples

The data from the quality control (QC) intra- and inter-assay Accuracy (RE) and Precision (CV) runs is shown in the tables below. The following QC sample concentrations for SEA-CD40 were prepared and analyzed over three runs: 0.500 ng/mL (LLOQ QC), 1.50 ng/mL (QC1), 25.0 ng/mL (QC3), 38.0 ng/mL (QC4). The maximum run size was run 7, which the length of run was assessed by extending over two 96-well plates (192 injections). Overall, the CV (%) for the intra-assay was ≤9.4% and ≤8.0% for the inter-assay, and the RE (%) for the intra-assay was −10.2% to 5.3% and −1.1% to −0.4% for the inter-assay. Acceptance was met for all quality control samples.

TABLE 10

| | | SEA-CD40 Concentration | | | |
|---|---|---|---|---|---|
| | | LLOQ QC | QC1 | QC3 | QC4 |
| | Run | 0.500 | 1.50 | 25.0 | 38.0 |
| | No. | ng/mL | ng/mL | ng/mL | ng/mL |
| | 1 | 0.483 | 1.46 | 25.1 | 39.0 |
| | | 0.539 | 1.47 | 25.3 | 38.1 |
| | | 0.515 | 1.54 | 24.5 | 41.8 |
| | | 0.539 | 1.56 | 25.7 | 39.7 |
| | | 0.534 | 1.41 | 25.7 | 37.2 |
| | | 0.546 | 1.39 | 25.2 | 39.4 |
| Intrarun Mean | | 0.526 | 1.47 | 25.3 | 39.2 |
| Intrarun SD | | | | | |
| Intrarun CV (%) | | | | | |
| Intrarun RE (%) | | | | | |
| n | | | | | |
| | | 0.0235 | 0.0679 | 0.446 | 1.57 |
| | | 4.5 | 4.6 | 1.8 | 4.0 |
| | | 5.2 | −2.0 | 1.2 | 3.2 |
| | | 6 | 6 | 6 | 6 |
| | 2 | 0.448 | 1.42 | 23.8 | 34.3 |
| | | 0.413 | 1.55 | 26.3 | 33.8 |
| | | 0.452 | 1.37 | 25.4 | 36.4 |
| | | 0.453 | 1.42 | 22.0 | 33.4 |
| | | 0.436 | 1.41 | 24.9 | 31.7 |
| | | 0.492 | 1.41 | 20.4 | 37.1 |
| Intrarun Mean | | 0.449 | 1.43 | 23.8 | 34.5 |
| Intrarun SD | | 0.0258 | 0.0616 | 2.23 | 2.00 |
| Intrarun CV (%) | | 5.7 | 4.3 | 9.4 | 5.8 |
| Intrarun RE (%) | | −10.2 | −4.7 | −4.8 | −9.2 |
| n | | 6 | 6 | 6 | 6 |
| | 7 | 0.510 | 1.40 | 23.1 | 39.9 |
| | | 0.499 | 1.54 | 27.1 | 35.2 |
| | | 0.510 | 1.49 | 24.2 | 39.5 |
| | | 0.531 | 1.76 | 28.5 | 39.1 |
| | | 0.508 | 1.55 | 27.7 | 38.8 |
| | | 0.501 | 1.74 | 24.0 | 42.8 |
| Intrarun Mean | | 0.510 | 1.58 | 25.8 | 39.2 |
| Intrarun SD | | 0.0114 | 0.142 | 2.27 | 2.44 |
| Intrarun CV (%) | | 2.2 | 9.0 | 8.8 | 6.2 |
| Intrarun RE (%) | | 2.0 | 5.3 | 3.2 | 3.2 |
| n | | 6 | 6 | 6 | 6 |
| Intrarun Mean | | 0.495 | 1.49 | 24.9 | 37.6 |
| Intrarun SD | | 0.0395 | 0.112 | 1.94 | 3.00 |
| Intrarun CV (%) | | 8.0 | 7.5 | 7.8 | 8.0 |
| Intrarun RE (%) | | −1.0 | −0.7 | −0.4 | −1.1 |
| n | | 18 | 18 | 18 | 18 |

TABLE 11

| SEA-CD40 | Intra-Run | Inter-run | Intra-Run | Inter-run |
|---|---|---|---|---|
| Concentration | CV (%) | CV (%) | RE (%) | RE (%) |
| LLOQ QC 0.500 | ≤5.7% | 8.0% | −10.2 to 5.2% | −1.0% |
| ng/mL | | | | |
| QC1 1.50 ng/mL | ≤9.0% | 7.5% | −4.7 to 5.3% | −0.7% |
| QC3 25.0 ng/mL | ≤9.4% | 7.8% | −4.8 to 3.2% | −0.4% |
| QC4 38.0 ng/mL | ≤6.2% | 8.0% | −9.2 to 3.2% | −1.1% |

Accuracy and Precision of Dilution Quality Control Samples

The data from the dilution quality control (QC) intra- and inter-assay Accuracy (RE) and Precision (CV) runs is shown in the table below. The dilution QC samples were prepared at a concentration of 250 ng/mL, 10-fol dilution, for SEA-CD40 and analyzed over three runs. Overall, the CV (%) for the intra-assay was <7.5% and 6.5% for the inter-assay, and the RE (%) for the intra-assay was -0.4% to 10.0% and 4.8% for the inter-assay. Acceptance was met for all dilution QC samples.

TABLE 12

| | | SEA-CD40 |
|---|---|---|
| | | Concentration |
| | Run | DilQC (10-fold dil.) |
| | Number | 250 ng/mL |
| | 1 | 257 |
| | | 261 |
| | | 256 |
| | | 262 |
| | | 279 |
| | | 261 |
| Intrarun Mean | | 263 |
| Intrarun SD | | 8.36 |
| Intrarun CV (%) | | 3.2 |
| Intrarun RE (%) | | 5.2 |
| n | | 6 |
| | 2 | 276 |
| | | 260 |
| | | 243 |
| | | 253 |
| | | 223 |
| | | 236 |
| Intrarun Mean | | 249 |
| Intrarun SD | | 18.7 |
| Intrarun CV (%) | | 7.5 |
| Intrarun RE (%) | | −0.4 |
| n | | 6 |
| | 7 | 284 |
| | | 257 |
| | | 280 |
| | | 276 |
| | | 289 |
| | | 266 |
| Intrarun Mean | | 275 |
| Intrarun SD | | 11.9 |
| Intrarun CV (%) | | 4.3 |
| Intrarun RE (%) | | 10.0 |
| n | | 6 |

### Summary

The LC/HRMS/MS method for the quantitation of SEA-CD40 in 200 μL of human plasma with K2EDTA anticoagulant detected a concentration range of 0.500 to 50.0 ng/mL of SEA-CD40, which is acceptable for use in clinical studies.

### Example 3: Tryptic Digestion of SEA-CD40 in Human Plasma

### 3.1 Trypsin Digest Conditions

Different tryptic digestion conditions were tested for use in the assay as described in Example 2. The standards used for each tryptic digestion condition include: Control blank, Zero Blank, 0.25 (STD 1), 0.50 (STD 2), 1.0 (STD 3), 2.0 (STD 4), 5.0 (STD 5), 10.0 (STD 6), 50.0 (STD 7) and 60 (STD 8) ng/mL SEA-CD40 in human plasma samples. Peptide extraction was conducted by immunoprecipitation. The following tryptic digestion conditions were tested:
1. 10:90 ACN:30 mM HCl elution+0.25 M Tris, pH 8.3+(no reduction and alkylation)+Tryptic digestion
2. 30 mM HCl elution+0.25 M Tris, pH 8.3+(no reduction and alkylation)+Tryptic digestion
3. 10:90 ACN:30 mM HCl elution+0.25 M Tris, pH 8.3+(no reduction and alkylation)+Urea denaturation+Tryptic digestion
4. 10:90 ACN:30 mM HCl elution+0.25 M Tris, pH 8.3+(no reduction and alkylation)+RapiGest (0.05%) denaturation+Tryptic digestion
5. 10:90 ACN:30 mM HCl elution+0.25 M Tris, pH 8.3+(no reduction and alkylation)+RapiGest (0.1%) denaturation+Tryptic digestion
6. 10:90 ACN:30 mM HCl elution+1.0 M Tris, pH 8.3+TCEP reduction+IAA alkylation+Tryptic digestion

### Results

The chromatograms for the control blank, zero blank and 0.50 ng/mL of the standard for condition 1 were obtained. The chromatograms for the control blank, zero blank and 0.50 ng/mL of the standard for condition 2 were obtained. The calibration curves for both conditions were determined.

The results for condition 1 and condition 2 indicate that a good signal can still be obtained at 0.50ng/mL without reduction and alkylation. By avoiding reduction and alkylation, the salt load overall can be reduced and translate to better robustness of the assay. Condition 2 provided the best signal (30mM HCl elution obtained a better signal than the 10:90 ACN:30 mM HCl elution). Adding the urea denaturation in condition 3 did not help the tryptic digestion for obtaining a better signal. Conditions 4 and 5, which utilized RapiGest denaturation, did not result in any signal.

### 3.2 Trypsin/Lys-C Digest Conditions

Digestion with trypsin/Lys-C was tested for use in the assay as described in Example 2. The standards used for the trypsin/Lys-C digestion include: Control blank, Zero Blank, 0.25 (STD 1), 0.50 (STD 2), 1.0 (STD 3), 2.0 (STD 4), 5.0 (STD 5), 10.0 (STD 6), 40.0 (STD 7) and 50 (STD 8) ng/mL SEA-CD40 in human plasma samples. Peptide extraction was conducted by immunoprecipitation on 200 μL of each of the plasma samples. No reduction and alkylation was conducted and 0.3 M Tris, pH 8.3 was used.

### Results

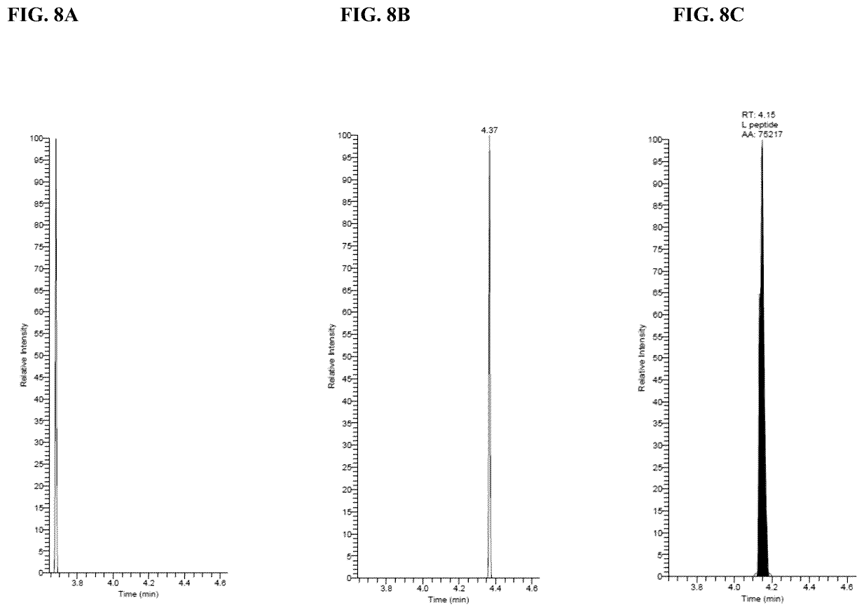

The chromatograms for the control blank, zero blank and 0.50 ng/mL of the standard (STD 2) are shown in FIGS. 8A-8C, respectively. The calibration curve was determined. The calibration curve statistics are shown in the table below. The quality control (QC) statistics are shown in Table 14. Based on the calibration curve and QC, trypsin/Lys-C digest provides a robust result for use in the assay.

TABLE 13

| | | Calibration | Conc. | Deviation |
|---|---|---|---|---|
| Sample ID | Rep. | Conc. (ng/mL) | Found | (%) |
| STD 1 | 1 | 0.25 | 0.274 | 9.6 |
| STD 1 | 2 | 0.25 | 0.393 | 57.2 |
| STD 2 | 1 | 0.5 | 0.385 | −23 |
| STD 2 | 2 | 0.5 | 0.622 | 24.4 |
| STD 3 | 1 | 1 | 0.9 | −10 |
| STD 3 | 2 | 1 | 1.09 | 9 |
| STD 4 | 1 | 2 | 1.81 | −9.5 |
| STD 4 | 2 | 2 | 2.09 | 4.5 |
| STD 5 | 1 | 5 | 4.81 | −3.8 |
| STD 5 | 2 | 5 | 5.41 | 8.2 |
| STD 6 | 1 | 10 | 9.64 | −3.6 |
| STD 6 | 2 | 10 | 10.2 | 2 |
| STD 7 | 1 | 40 | 6.85 | −82.9 |
| STD 7 | 2 | 40 | 39.7 | −0.8 |
| STD 8 | 1 | 50 | 50.7 | 1.4 |
| STD 8 | 2 | 50 | 50.5 | 1 |

TABLE 14

| | LLOQ QC | LOQ | QC 1 | QC 2 | QC 3 |
|---|---|---|---|---|---|
| Theor. Conc. | 0.5 | 1.5 | 5 | 30 | 40 |
| (ng/mL) | | | | | |
| #1 | 0.599 | 1.76 | 5.2 | 31.8 | 41.1 |
| #2 | 0.531 | 1.54 | 5.5 | 31.6 | 40.4 |
| #3 | 0.572 | 1.51 | 5.37 | 31.3 | 43.1 |
| Mean | 0.567 | 1.6 | 5.36 | 31.6 | 41.5 |
| S.D. | 0.0342 | 0.137 | 0.15 | 0.252 | 1.4 |
| % CV | 6 | 8.6 | 2.8 | 0.8 | 3.4 |
| % Theoretical | 113.4 | 106.7 | 107.2 | 105.3 | 103.8 |
| % Dev | 13.4 | 6.7 | 7.2 | 5.3 | 3.8 |
| n | 3 | 3 | 3 | 3 | 3 |

In addition, a comparison was conducted between selected reaction monitoring (SRM) spectrometry and high-resolution mass spectrometry (HRMS) methods. The results suggested that HRMS MS2 provided a cleaner extracted ion chromatogram than SRM.

### Example 4: PK Studies in a Phase 1 Dose-Escalation Study of SEA-CD40

Pharmacokinetics (PK) studies were performed in a first-in-human phase 1 trial of SEA-CD40 monotherapy.

In the clinical trial, SEA-CD40 was administered intravenously (IV) on a 21-day cycle with doses ranging from 0.6-60 μg/kg with standard 3+3 dose escalation. SEA-CD40 was administered to 56 and 11 participants with solid tumors and lymphoma, respectively. More specifically, SEA-CD40 was administered IV in 21-day cycles (Day 1 of each cycle; Q3wk) with standard 3+3 dose escalation. The standard dosing regimen was 0.6, 3, 10, 30, 45, or 60 μg/kg on Day 1. An intensified dosing regimen was also examined, consisting of 30 μg/kg dosed on Day 1 and Day 8 of the first 2 cycles, with only one dose of SEA-CD40 administered on Day 1 in subsequent cycles.

Plasma samples for intensive PK testing were collected in Cycles 1, 2, and 4 in the dose escalation cohorts. Pre-dose samples were collected in Cycles 3, 5, and subsequent dosing cycles at times specified per protocol. SEA-CD40 plasma concentrations were analyzed via a validated liquid chromatography-mass spectroscopy/mass spectroscopy assay as described in the present disclosure, with the lowest level of quantitation concentration of 0.5 ng/mL. PK parameter estimates for SEA-CD40 dosed as monotherapy were available for 41 participants with solid tumor malignancies (N=31 in dose escalation) and for 10 participants with lymphoma. Noncompartmental analysis was performed using Phoenix WinNonlin version 8.2 (Certara USA, Inc., Princeton, NJ) to determine PK parameters for each participant. Plasma concentration-time profiles and dose proportionality analyses were performed using GraphPad Prism version 8.0 (GraphPad Software Inc., San Diego, CA).

56 participants with solid tumors and 11 participants with lymphoma were enrolled. Enrollment by dose level between 0.6 to 60 μg/kg is shown in Error! Reference source not found.6.

TABLE 16
SEA-CD40 IV Monotherapy dose levels
and number of participants

| | | Solid | |
|---|---|---|---|
| | Dose Q3wk | Tumors | Lymphomas |
| | (Day 1 μg/kg) | (N) | (N) |
| Single-participant | 0.6 | 1 | 0 |
| cohorts | 3 | 1 | 0 |
| Standard 3 + 3 dose | 10 | 4 | 3 |
| escalation cohorts | 30 | 21 | 3 |
| | 45 | 6 | 3 |
| | 60 | 11 | 2 |
| | 30* | 12 | 0 |
| | Total | 56 | 11 |

*Intensified dosing (Days 1 and 8 in cycles 1 and 2, with Day 1 only in subsequent cycles)

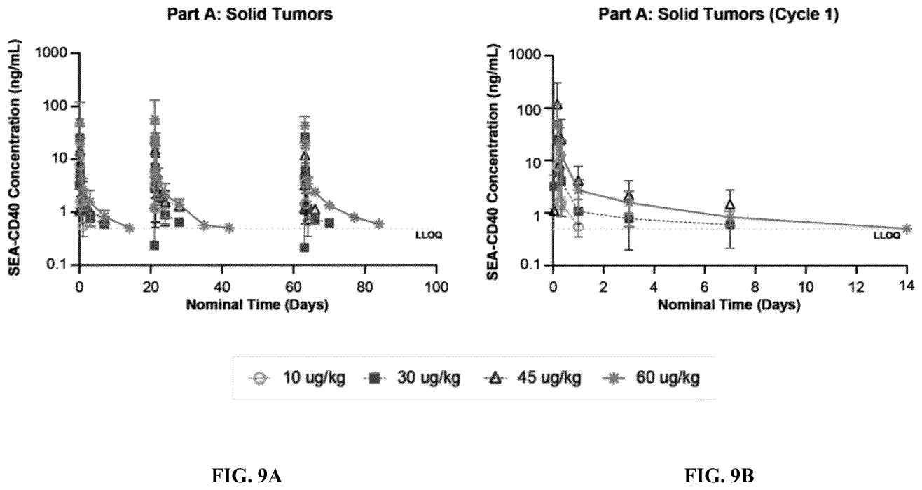

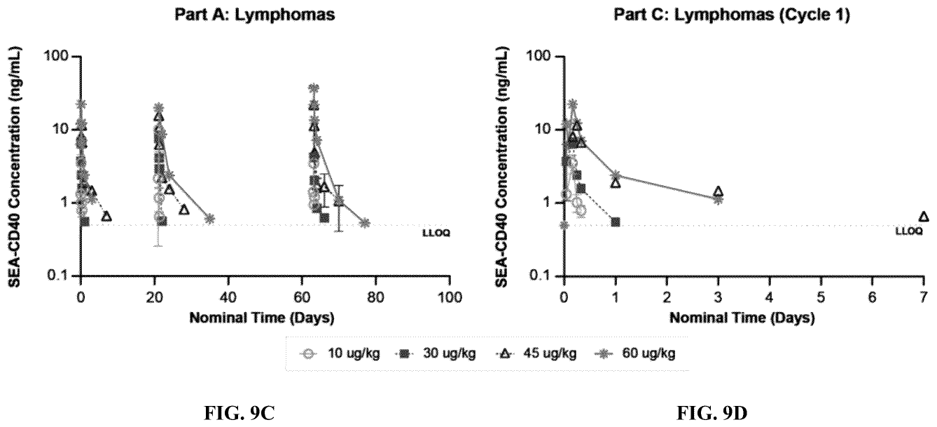

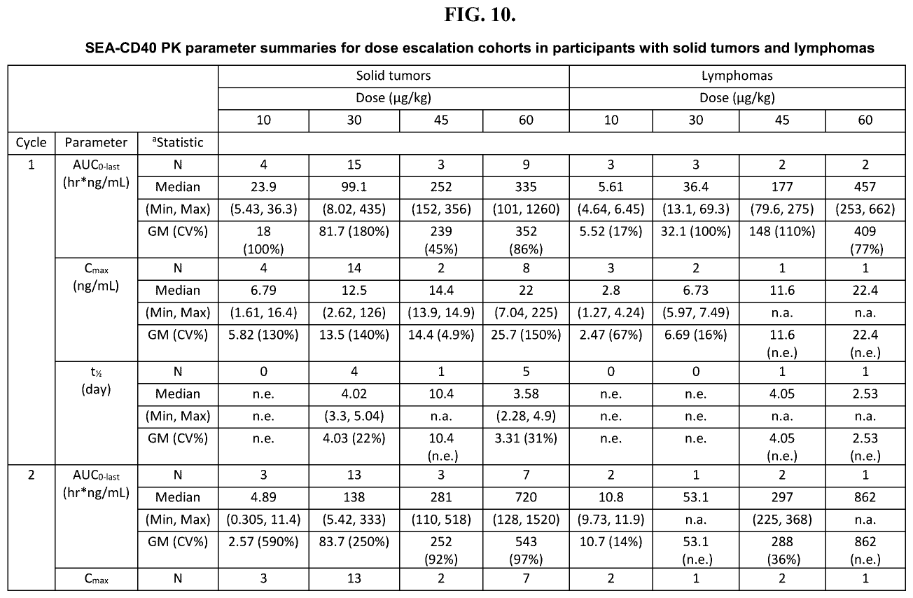

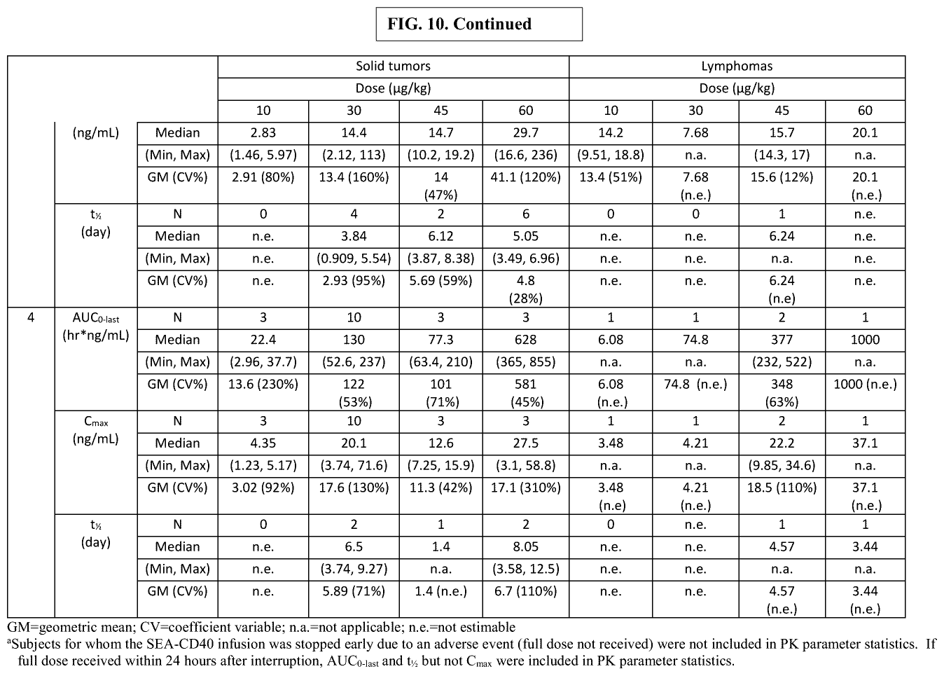

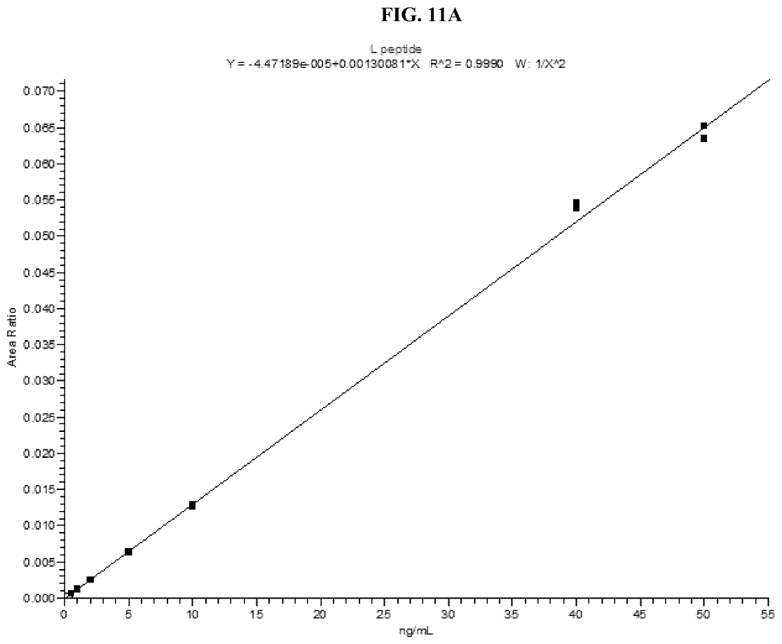

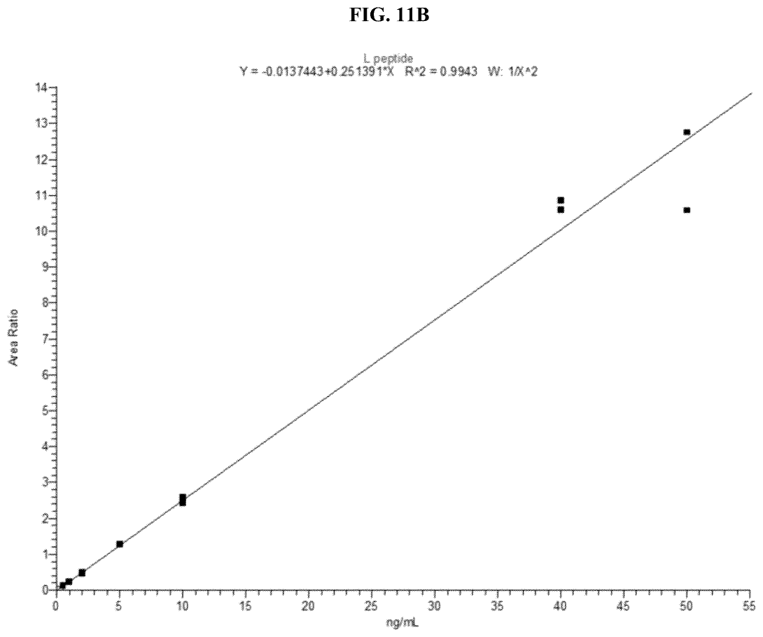

PK was analyzed in participants who received SEA-CD40 at doses of 10, 30, 45, or 60 μg/kg by IV infusion of variable duration (3 to 613 min). The arithmetic mean (standard deviation) SEA-CD40 serum concentration versus time profiles for 10-60 μg/kg SEA-CD40 monotherapy are shown in FIGS. 9A-9D. At the lowest SEA-CD40 dose levels tested (0.6 and 3 μg/kg), a majority of serum concentrations were below the lower limit of quantitation (0.500 ng/mL) which precluded estimation of PK parameters. Estimated PK parameter summaries for the other dose levels are shown in FIG. 10.

Average area under the concentration-time curve from time zero to time of last measurable concentration (AUC₀₋ₗₐₛₜ) values were greater than dose proportional below 30 μg/kg and approximately dose-proportional from 30 to 60 μg/kg in the solid tumor dose escalation cohort and greater than dose proportional from 10 to 60 μg/kg in the lymphoma dose escalation cohort. IV infusion rates and lengths in Cycles 1, 2, and 4 were highly variable; therefore, dose proportionality was not assessed using Cmax due to these variabilities. The SEA-CD40 Cmax was attained at the end of the infusion, after which serum concentrations decreased rapidly over time in a multi-exponential fashion.

Median terminal half-life (ty2) estimations for SEA-CD40 in the solid tumor dose escalation cohort were 4.0 (n=4), 10.4 (n=1), and 3.6 (n=5) days after administration of 30, 45, or 60 !As/kg, respectively (Cycle 1) (FIG. 10). In all cohorts, most subjects did not have detectable SEA-CD40 by pre-dose of the subsequent cycle. The median and geometric mean (GM) exposures were higher in the solid tumor cohort compared to the lymphoma cohort from 10 to 45 μg/kg doses, although given the low subject numbers in the lymphoma cohort this observation should be interpreted with caution. SEA-CD40 exposures were of similar magnitudes after Q3wk treatment Cycles 1, 2, and 4, suggesting that SEA-CD40 did not accumulate upon repeat dosing (FIGS. 9A-9D and 10). Dose intensification resulted in similar serum concentration profiles, with no evidence of accumulation upon repeat dosing.

The results showed that SEA-CD40 was adequately tolerated in participants with advanced solid tumors and lymphoma. Evidence of robust pharmacodynamic activity was observed in both solid tumor and lymphoma participants.

### Example 5: Stable Isotope Labeling by/with Amino Acids in Cell Culture (SILAC)

SEA-CD40 was expressed in the presence of isotope-labeled amino acid residues. A pre-determined amount of isotope-labeled SEA-CD40 was added to calibration solutions and quality control solutions. The labeled and unlabeled SEA-CD40 were extracted using the same immunoprecipitation extraction procedure, and were treated with trypsin digest solution and analyzed by the methods as described in Example 2. Because of the presence of isotopes in the labeled SEA-CD40, the isotopes added a mass shift to the same analytic peptides derived from the labeled SEA-CD40. The peak of the analytic peptides from the labeled and unlabeled were compared against each other.

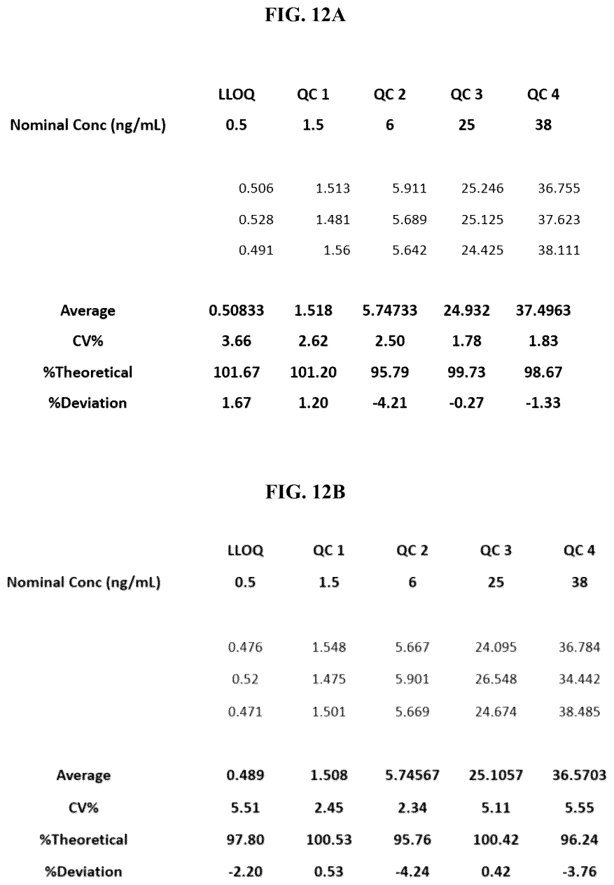

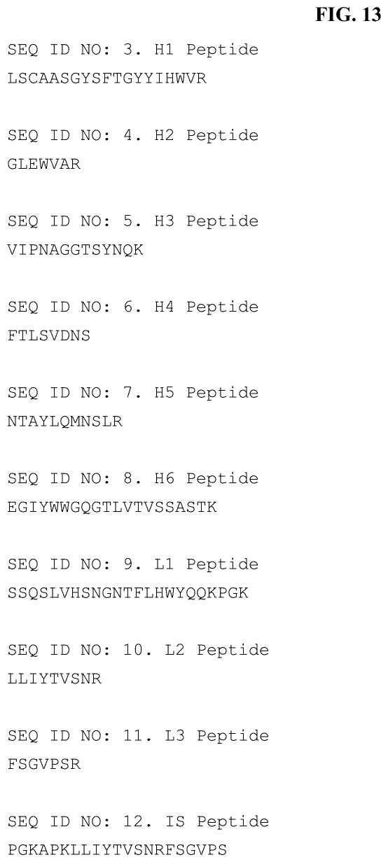

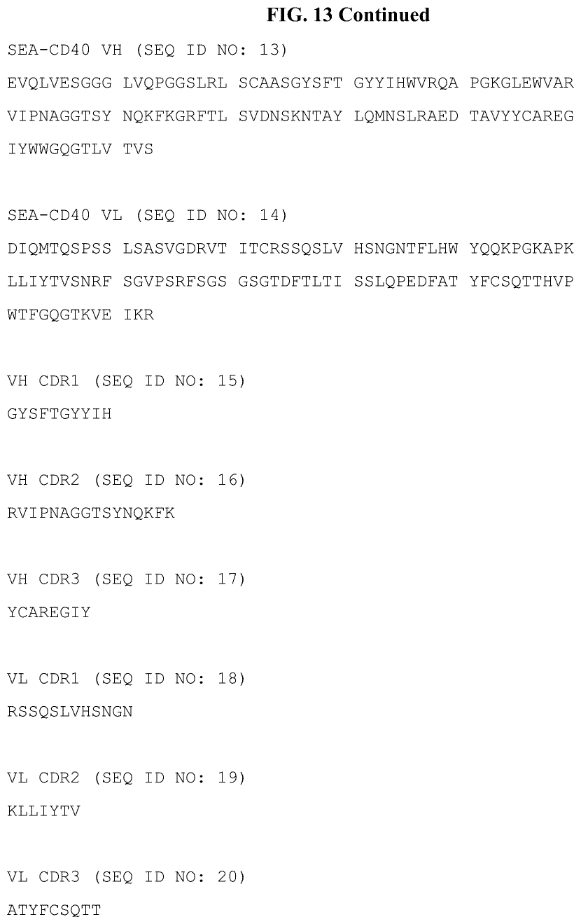

The peak area for an analytic peptide was divided by the peak area for the same analytic peptide derived from the isotope labeled SEA-CD40, and the resultant peak area ratios were plotted as a function of standard concentrations for standard curve samples. The data points was fitted to a linear regression analysis. The results are shown in FIG. 11A. In addition, the results from quality control samples were also analyzed. The results are shown in FIG. 12A. In contrast, the calibration curve using the internal standard is shown in FIG. 11B. The results from quality control samples using the internal standard is shown in FIG. 12B. The results suggest that the methods using SILAC further improves accuracy.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the amount of an anti-CD40 antibody or antigen-binding fragment thereof in a sample, wherein the anti-CD40 antibody or antigen-binding fragment thereof comprises: a heavy chain variable region (VH) comprising an amino acid sequence of SEQ ID NO: 13; and a light chain variable region (VL) comprising an amino acid sequence of SEQ ID NO: 14, the method comprising
extracting the anti-CD40 antibody or antigen-binding fragment thereof from the sample;
digesting the extracted anti-CD40 antibody or antigen-binding fragment thereof with a protease to release one or more analytic peptides from the anti-CD40 antibody or antigen-binding fragment thereof; and
detecting the one or more analytic peptides by mass spectrometry, thereby determining the amount of the anti-CD40 antibody or antigen-binding fragment thereof in the sample.

2. The method of claim 1, wherein the anti-CD40 antibody or antigen-binding fragment thereof is extracted by anti-idiotypic affinity capture.

3. The method of claim 1 or 2, wherein the extracted anti-CD40 antibody or antigen-binding fragment thereof is digested by either one or both of trypsin and Lys-C.

4. The method of any one of claims 1-3, wherein the extracted anti-CD40 antibody or antigen-binding fragment thereof is digested in a digestion solution comprising about 10 μg/mL to about 100 μg/mL of trypsin and/or Lys-C.

5. The method of claim 4, wherein the concentration of trypsin and/or Lys-C in the digest solution is about 50 μg/mL

6. The method of claim 4 or 5, wherein the digestion solution further comprises about 0.25 M to about 1.0 M Tris, with a pH of about 8.0 to about 8.5.

7. The method of any one of claims 1-6, wherein the extracted anti-CD40 antibody or antigen-binding fragment thereof is not subject to either reduction or alkylation.

8. The method of any one of claims 2-7, wherein the extracted anti-CD40 antibody or antigen-binding fragment thereof is eluted from the anti-idiotypic affinity capture with an elution solution comprising about 10 mM to about 50 mM hydrochloric acid (HCl).

9. The method of claim 8, wherein the extracted anti-CD40 antibody or antigen-binding fragment thereof is eluted from the anti-idiotypic affinity capture with an elution solution comprising about 30 mM hydrochloric acid (HCl).

10. The method of any one of claims 1-9, wherein the anti-CD40 antibody comprises a human constant region.

11. The method of any one of claims 1-10, wherein the anti-CD40 antibody comprises a heavy chain with a sequence set forth in SEQ ID NO: 1 and a light chain with a sequence set forth in SEQ ID NO: 2.

12. The method of any one of claim 1-11, wherein the anti-CD40 antibody is non-fucosylated.

13. The method of any one of claim 1-12, wherein the anti-CD40 antibody is SEA-CD40.

14. The method of any one of claims 1-11, wherein less than 5% of the anti-CD40 antibody in the sample has an N-glycoside-linked sugar chain that comprises a fucose residue at residue N297 according to the EU numbering.

15. The method of any one of claims 1-14, wherein at least one of the one or more analytic peptides comprises at least one amino acid residue from a CDR of the anti-CD40 antibody or the antigen-binding fragment thereof.

16. The method of any one of claims 1-15, wherein at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11).

17. The method of claim 16, wherein at least one of the one or more analytic peptides comprises the amino acid sequence of LLIYTVSNR (SEQ ID NO: 10).

18. The method of any one of claims 1-17, wherein the sample is plasma or serum.

19. The method of claim 18, wherein the plasma sample is treated with an anticoagulant.

20. The method of claim 19, wherein the anticoagulant is dipotassium ethylenediaminetetraacetic acid (K2EDTA).

21. The method of any one of claims 1-20, wherein the sample is from a human patient that has been administered the anti-CD40 antibody or antigen-binding fragment thereof

22. The method of any one of claims 1-21, wherein the sample is at least 200 μL.

23. The method of any one of claims 1-22, wherein the method has a lower limit of quantification (LLOQ) of about 0.1 ng/mL to about 1 ng/ml.

24. The method of claim 23, wherein the method has a lower limit of quantification (LLOQ) of about 0.5 ng/mL.

25. The method of any one of claims 1-24, wherein the one or more analytic peptides are detected by a liquid chromatography tandem mass spectrometry (LC-MS/MS) system.

26. The method of claim 25, wherein the LC-MS/MS system comprises a trap column and a nano column.

27. The method of claim 26, wherein the trap column and the nano column are run at a temperature between about 50° C. to about 70° C.

28. The method of any one of claims 25-27, wherein nano-electrospray ionization with high resolution (LC-HRMS/MS) is used in the LC-MS/MS system.

29. The method of claim 28, wherein the LC-HRMS/MS system is in the positive ion mode.

30. The method of any one of claims 1-29, wherein the anti-CD40 antibody or antigen-binding fragment thereof has a concentration of about 0.5 ng/mL to about 50.0 ng/mL in the sample.

31. The method of any one of claims 1-30, wherein an isotope-labeled peptide is added to the sample before digesting the extracted anti-CD40 antibody or antigen-binding fragment thereof

32. The method of claim 31, wherein the isotope-labeled peptide is an isotope-labeled anti-CD40 antibody or antigen-binding fragment thereof.

33. The method of claim 31 or 32, wherein the isotope-labeled peptide comprises PGKAPKLLIYTV\hat{ }SNR\hat{ }FSGVPS (SEQ ID NO: 12), wherein V\hat{ } represents D C and/or ¹⁵N labeled valine, and R\hat{ } represents D C and/or ¹⁵N labeled arginine.

34. A method for determining the amount of an anti-CD40 antibody or antigen-binding fragment thereof in a sample, wherein the anti-CD40 antibody or antigen-binding fragment thereof comprises: a heavy chain variable region (VH) comprising an amino acid sequence of SEQ ID NO: 13; and a light chain variable region (VL) comprising an amino acid sequence of SEQ ID NO: 14; the method comprising extracting the anti-CD40 antibody or antigen-binding fragment thereof from the sample;
mixing a fixed amount of an isotope-labeled peptide with the extracted anti-CD40 antibody or antigen-binding fragment thereof ;
digesting the extracted anti-CD40 antibody or antigen-binding fragment thereof and the isotope-labeled peptide with a protease to release one or more analytic peptides from the anti-CD40 antibody or antigen-binding fragment thereof and one or more isotope-labeled analytic peptide fragments from the isotope-labeled peptide; and
detecting the one or more analytic peptides and the one or more isotope-labeled analytic peptide fragments by mass spectrometry, thereby determining the amount of the anti-CD40 antibody or antigen-binding fragment thereof in the sample.

35. The method of claim 34, wherein the isotope-labeled peptide is an isotope-labeled anti-CD40 antibody or antigen-binding fragment thereof.

36. The method of claim 34 or 35, wherein the isotope-labeled peptide comprises PGKAPKLLIYTV\hat{ }SNR\hat{ }FSGVPS (SEQ ID NO: 12), wherein V\hat{ } represents ¹³C and/or ¹⁵N labeled valine, and R\hat{ } represents D C and/or ¹⁵N labeled arginine.

37. The method of any one of claims 34-36, wherein the anti-CD40 antibody or antigen-binding fragment thereof is extracted by anti-idiotypic affinity capture.

38. The method of any one of claims 34-37, wherein the extracted anti-CD40 antibody or antigen-binding fragment thereof is digested by either one or both of trypsin and Lys-C.

39. The method of any one of claims 34-38, wherein the anti-CD40 antibody comprises a human constant region.

40. The method of any one of claims 34-39, wherein the anti-CD40 antibody is non-fucosylated.

41. The method of any one of claims 34-40, wherein the anti-CD40 antibody is SEA-CD40.

42. The method of any one of claims 34-41, wherein less than 5% of the anti-CD40 antibody in the sample has an N-glycoside-linked sugar chain that comprises a fucose residue at residue N297 according to the EU numbering.

43. The method of any one of claims 34-42, wherein at least one of the one or more analytic peptides comprises at least one amino acid residue from a CDR of the anti-CD40 antibody or the antigen-binding fragment thereof.

44. The method of any one of claims 34-43, wherein at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11).

45. The method of claim 44, wherein at least one of the one or more analytic peptides comprises the amino acid sequence of LLIYTVSNR (SEQ ID NO: 10).

46. The method of any one of claims 34-45, wherein the method has a lower limit of quantification (LLOQ) of about 0.1 ng/mL to 1 ng/ml.

47. The method of claim 46, wherein the method has a lower limit of quantification (LLOQ) of about 0.5 ng/mL.

48. The method of any one of claims 34-47, wherein the one or more analytic peptides are detected by a liquid chromatography tandem mass spectrometry (LC-MS/MS).

49. A method for determining the amount of an anti-CD40 antibody or antigen-binding fragment thereof in a sample, wherein the anti-CD40 antibody or antigen-binding fragment thereof comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2; the method comprising
adding a fixed amount of an isotope-labeled antibody or antigen-binding fragment thereof to the sample, wherein the isotope-labeled antibody or antigen-binding fragment thereof comprises VH CDR1, VH CDR2, VH CDR3 of SEQ ID NO: 1 and VL CDR1, VL CDR2, VL CDR3 of SEQ ID NO: 2;
extracting the anti-CD40 antibody or antigen-binding fragment thereof and the isotope-labeled antibody or antigen-binding fragment thereof from the sample;
digesting the extracted anti-CD40 antibody or antigen-binding fragment thereof and the extracted isotope-labeled antibody or antigen-binding fragment thereof with a protease to release one or more analytic peptides from the anti-CD40 antibody or antigen-binding fragment thereof and one or more isotope-labeled peptides from the isotope-labeled antibody or antigen-binding fragment thereof; and
detecting the one or more analytic peptides and the one or more isotope-labeled peptides by mass spectrometry, thereby determining the amount of the anti-CD40 antibody or antigen-binding fragment thereof in the sample.

50. The method of claim 49, wherein the isotope-labeled antibody or antigen-binding fragment thereof comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2.

51. The method of claim 50, wherein the isotope-labeled antibody or antigen-binding fragment thereof comprises a heavy chain variable region comprising amino acids 1-113 of SEQ ID NO: 1 and a light chain variable region comprising amino acids 1-113 of SEQ ID NO: 2.

52. The method of any one of claims 49-51, wherein the isotope-labeled antibody comprises a human constant region.

53. The method of any one of claim 49-52, wherein the isotope-labeled antibody is non-fucosylated.

54. The method of any one of claims 49-51, wherein the isotope-labeled antibody is an isotope-labeled SEA-CD40.

55. The method of any one of claims 49-51, wherein less than 5% of isotope-labeled antibody in the fixed amount of an isotope-labeled antibody or antigen-binding fragment thereof has an N-glycoside-linked sugar chain that comprises a fucose residue at residue N297 according to the EU numbering.

56. The method of any one of claims 49-55, wherein at least one of the one or more isotope-labeled peptides comprises at least one amino acid residue from a CDR of the isotope-labeled antibody or antigen-binding fragment thereof.

57. The method of any one of claims 49-56, wherein at least one of the one or more isotope-labeled peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11).

58. The method of claim 57, wherein at least one of the one or more isotope-labeled peptides comprises the amino acid sequence of LLIYTVSNR (SEQ ID NO: 10).

59. The method of any one of claims 49-58, wherein the anti-CD40 antibody or antigen-binding fragment thereof and the isotope-labeled antibody or antigen-binding fragment thereof are extracted by anti-idiotypic affinity capture.

60. The method of any one of claims 49-59, wherein the extracted anti-CD40 antibody or antigen-binding fragment thereof and the isotope-labeled antibody or antigen-binding fragment thereof are digested by either one or both of trypsin and Lys-C.

61. The method of any one of claims 49-60, wherein the anti-CD40 antibody comprises a human constant region.

62. The method of any one of claim 49-61, wherein the anti-CD40 antibody is non-fucosylated.

63. The method of any one of claim 49-62, wherein the anti-CD40 antibody is SEA-CD40.

64. The method of any one of claims 49-63, wherein less than 5% of the anti-CD40 antibody or antigen-binding fragment thereof in the sample has an N-glycoside-linked sugar chain that comprises a fucose residue at residue N297 according to the EU numbering.

65. The method of any one of claims 49-64, wherein at least one of the one or more analytic peptides comprises at least one amino acid residue from a CDR of the anti-CD40 antibody or the antigen-binding fragment thereof.

66. The method of any one of claims 49-65, wherein at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO: 6), LLIYTVSNR (SEQ ID NO: 10), and FSGVPSR (SEQ ID NO: 11).

67. The method of claim 66, wherein at least one of the one or more analytic peptides comprises the amino acid sequence of LLIYTVSNR (SEQ ID NO: 10).

68. The method of any one of claims 49-67, wherein the method has a lower limit of quantification (LLOQ) of about 0.1 ng/mL to 1 ng/ml.

69. The method of claim 68, wherein the method has a lower limit of quantification (LLOQ) of about 0.5 ng/mL.

70. The method of any one of claims 49-69, wherein the one or more analytic peptides are detected by a liquid chromatography tandem mass spectrometry (LC-MS/MS).

71. A method for determining the amount of an anti-CD40 antibody in a plasma sample, wherein the anti-CD40 antibody comprises: a heavy chain variable region (VH) comprising an amino acid sequence that is identical to VH in a heavy chain of SEQ ID NO: 1; and a light chain variable region (VL) comprising an amino acid sequence that is identical to VL in a light chain of SEQ ID NO: 2; and a human constant region; the method comprising
a) extracting the anti-CD40 antibody from the sample by anti-idiotypic affinity capture, wherein the plasma sample is obtained from a subject after being administered with a composition comprising the anti-CD40 antibody, wherein less than 5% of the anti-CD40 antibody in the composition has an N-glycoside-linked sugar chain that comprises a fucose residue at residue N297 according to the EU numbering;
b) digesting the extracted anti-CD40 antibody with one or both of trypsin and Lys-C to release one or more analytic peptides from the anti-CD40 antibody; and
c) detecting the one or more analytic peptides by a nanoscale liquid chromatography tandem mass spectrometry (nano LC-MS/MS), thereby determining the amount of the anti-CD40 antibody in the plasma sample.

72. The method of claim 71, further comprising:
adding a fixed amount of an isotope-labeled peptide to the sample before digesting the extracted anti-CD40 antibody.

73. The method of claim 71 or 72, wherein at least one of the one or more analytic peptides comprises an amino acid sequence selected from LSCAASGYSFTGYYIHWVR (SEQ ID NO: 3), GLEWVAR (SEQ ID NO: 4), VIPNAGGTSYNQK (SEQ ID NO: 5), FTLSVDNS (SEQ ID NO:

6. , LLIYTVSNR (SEQ ID NO: 10), and FSGVPSR (SEQ ID NO: 11).

74. The method of claim 73, wherein at least one of the one or more analytic peptides comprises the amino acid sequence of LLIYTVSNR (SEQ ID NO: 10).

75. The method of any one of claims 71-74, wherein the method has a lower limit of quantification (LLOQ) of about 0.1 ng/mL to 1 ng/ml.

76. The method of claim 75, wherein the method has a lower limit of quantification (LLOQ) of about 0.5 ng/mL.

77. A peptide or peptide fragment comprising an amino acid sequence selected from
(SEQ ID NO: 3)
LSCAASGYSFTGYYIHWVR,
(SEQ ID NO: 4)
GLEWVAR,
(SEQ ID NO: 5)
VIPNAGGTSYNQK,
(SEQ ID NO: 6)
FTLSVDNS,
(SEQ ID NO: 7)
NTAYLQMNSLR,
(SEQ ID NO: 8)
EGIYWWGQGTLVTVSSASTK,
(SEQ ID NO: 9)
SSQSLVHSNGNTFLHWYQQKPGK,
(SEQ ID NO: 10)
LLIYTVSNR,
and
(SEQ ID NO: 11)
FSGVPSR.

78. The peptide or peptide fragment of claim 77, wherein peptide or peptide fragment comprises the amino acid sequence LLIYTVSNR (SEQ ID NO: 10).

79. The peptide or peptide fragment of claim 77 or 78, wherein the peptide or peptide fragment is used in a method for determining the amount of an anti-CD40 antibody in a sample.

80. A peptide or peptide fragment comprising the amino acid sequence
(SEQ ID NO: 12)
PGKAPKLLIYTVSNRFSGVPS.

81. The peptide or peptide fragment of claim 80, wherein the peptide or peptide fragment comprises at least one isotopic label.

82. The peptide or peptide fragment of claim 81, wherein the at least one isotopic label is selected from ¹³C and ¹⁵N.

83. The peptide or peptide fragment of any one of claims 80-82, wherein the peptide or peptide fragment comprises PGKAPKLLIYTV\hat{ }SNR\hat{ }FSGVPS, wherein V\hat{ } represents D C and/or ¹⁵N labeled valine, and R\hat{ } represents ¹³C and/or ¹⁵N labeled arginine.
